(19) United States
(12) Patent Application Publication
(10) Pub. No.: US 2024/0090795 A1
(43) Pub. Date: Mar. 21, 2024

(54) Methods for Pulmonary Function Testing With Machine Learning Analysis and Systems for Same

(71) Applicant: The Regents of the University of California, Oakland, CA (US)

(72) Inventors: Franklin Heng, San Francisco, CA (US); Xavier Minh Mathieu Orain, San Francisco, CA (US)

(21) Appl. No.: 18/275,170

(22) PCT Filed: Feb. 2, 2022
(86) PCT No.: PCT/US2022/014922

§ 371 Date: Jul. 31, 2023

Related U.S. Application Data
(60) Provisional application No. 63/145,106, filed on Feb. 3, 2021.

(51) Int. Cl. A61B 5/091; A61B 5/00; A61B 5/087; G06T 7/00; G06T 7/246; G06V 10/25; G16H 30/40; G16H 50/30

(52) U.S. Cl. CPC A61B 5/091; A61B 5/087; A61B 5/7275; A61B 5/742; A61B 5/7485; G06T 7/0016; G06T 7/248; G06V 10/25; G16H 30/40; G16H 50/30; G06T 2207/10028; G06T 2207/30061; G06V 2201/031

(57) ABSTRACT

Methods and systems for pulmonary function testing of a subject are provided. Aspects of the present invention include methods and systems configured to generate flow volume curves and compute lung function parameters of a subject and determine potential clinical interpretations of pulmonary function. In addition, the present invention offers advantages including (i) measuring lung function without initial calibration of spirometer information, (ii) the ability to use spirometer information to develop a machine learning based algorithm which will eventually measure lung function without needing spirometer information at all, (iii) computing metrics such as chest and waist width and sitting height of subject.

### INTRODUCTION

Current methods available for physicians to measure patient lung function entails a procedure known as spirometry. Spirometry measures how well a patient's lungs work by having the patient perform strenuous breathing tasks through a tubular, electronic device called a spirometer. Global guidelines require the use of spirometry-based techniques to diagnose patients with certain chronic respiratory diseases. However, obtaining accurate results through existing spirometry-based techniques depends on, among other things, very rigorous coaching of the patient's breathing maneuvers and ideal testing conditions. In some cases, existing techniques lack clear and understandable feedback, making them unreliable as they do not always offer a well-standardized approach. For example, it is estimated that about 43% of primary care physicians have a spirometer in office but only a fraction of those use it. See O'Dowd, et al. Attitudes of physicians toward objective measures of airway function in asthma. The American Journal of Medicine, 114(5), 391-396. This can lead to significant mistreatment and misdiagnosis of patients with respiratory diseases such as, for example, asthma and chronic obstructive pulmonary disease (COPD).

### SUMMARY

Thus, there is a need for improved and useful methods and systems for assessing pulmonary function of patients. This invention provides such new and useful methods and systems. For example, embodiments of the present invention address limitations of current spirometry-based approaches to assessing lung function by: (1) performing lung function testing and analysis using only small depth camera sensors, thereby eliminating the need for spirometers and other disposable equipment; (2) providing real-time feedback and coaching to assist both the patient and physician; and (3) providing easily readable interpretation and diagnosis of the results of lung function assessments. Embodiments of the present invention will contribute to making lung function testing a more accessible and cost-efficient medical test in primary care, thereby improving patient outcomes.

In addition, the present invention offers advantages including
(i) measuring lung function without initial calibration of spirometer information,
(ii) the ability to use spirometer information to develop a machine learning based algorithm which will eventually measure lung function without needing spirometer information at all, (iii) computing metrics such as chest and waist width and sitting height of subject, (iv) capturing various subject health record information and technician coaching information to aid and improve algorithms according to the present invention, (v) the ability collect and utilize a higher volume of more diverse data points for utilization by algorithms according to the present invention, (vi) implementing skeleton tracking in methods according to the present invention, which will detect common position of subject and movement errors in practice; enabling such features allow better and more standardized results of lung function measures, (vii) incorporating automated interpretation along with lung function parameter results; embodiments of the present invention use graphs and discrete metrics to provide clinicians with clinically valuable analysis and (viii) the ability to observe multiple areas of a subject's body, instead of a single region of interest, in order to compute displacement graphs.

Methods and systems for pulmonary function testing of a subject are provided. Aspects of the present invention include systems comprising a first depth-sensing camera configured to generate depth-sensing images of a subject; and a processor comprising memory operably coupled to the processor, wherein the memory comprises instructions stored thereon, which, when executed by the processor, cause the processor to: identify, based on a reference image received from the depth-sensing camera, reference locations of certain features of interest on the subject; determine a chest region of interest comprising a chest area of the subject, based on the location of the features of interest; receive a plurality of images of the subject from the depth-sensing camera while the subject performs specified breathing maneuvers; generate a three-dimensional representation of the chest region of interest based on the plurality of images of the subject; compute changes in the volume of the chest region of interest based on the three-dimensional representation of the chest; plot the changes in volume of the chest region of interest on a graph, wherein the graph comprises volume of the chest region of interest over time and wherein certain chest movements are labeled on the graph; filter the data on the graph using one or more specified filters; generate a flow volume curve based at least in part on rescaling the filtered graph and computing a gradient of the rescaled graph; compute lung function parameters based at least in part on the flow volume curve; and determine potential clinical interpretations of the pulmonary function of the subject based at least in part on the computed lung function parameters; and an operable connection between the depth-sensing camera and the processor. Also provided are methods of performing pulmonary function testing of a subject. The methods and systems find use in a variety of different applications, e.g., the diagnosis and treatment of subjects with a respiratory disease, such as, for example, asthma or chronic obstructive pulmonary disease (COPD), subjects potentially in need of artificial ventilation including mechanical ventilation, such as, for example, subjects with amyotrophic lateral sclerosis (ALS).

### BRIEF DESCRIPTION OF THE FIGURES

The invention may be best understood from the following detailed description when read in conjunction with the accompanying drawings. Included in the drawings are the following figures:

FIG. 1A and FIG. 1B illustrate a flow diagram for conducting pulmonary function testing according to embodiments of the present invention. FIG. 1A comprises a flow diagram for computing a chest measures aspect of the invention, and FIG. 1B comprises a flow diagram for computing a lung parameters aspect of the present invention.

FIG. 2 depicts a flow diagram for computing motion and interpreting movements of a subject according to some aspects of the present disclosure.

FIG. 3 depicts a flow diagram for processing depth information and computing chest volume of a subject according to some aspects of the present disclosure.

FIG. 4 depicts a flow diagram for computing a quality of effort by a subject when performing breathing maneuvers used to evaluate the pulmonary function of the subject, according to some aspects of the present disclosure.

FIG. 5 depicts a flow diagram for computing a relationship between depth sensor information (and other data) and a subject's lung function, in order to translate chest displacement to lung function parameters, according to some aspects of the present disclosure.

FIGS. 6A and 6B depict an example of a subject utilizing a system according to the present disclosure to assess the subject's pulmonary lung function.

FIG. 7 depicts an example application of an embodiment of a subject method for pulmonary function testing of a subject according to the present disclosure.

FIG. 8A and FIG. 8B illustrate an alternative flow diagram for conducting pulmonary function testing according to embodiments of the present invention that is a variation of that depicted in FIG. 1A and FIG. 1B. FIG. 8A comprises a flow diagram for computing a chest measures aspect of the invention, and FIG. 8B comprises a flow diagram for computing a lung parameters aspect of the present invention.

FIG. 9 depicts an alternative flow diagram for processing depth information and computing chest volume of a subject according to some aspects of the present disclosure that is a variation of that depicted in FIG. 3.

FIG. 10 depicts an alternative flow diagram for computing a relationship between depth sensor information (and other data) and a subject's lung function, in order to translate chest displacement to lung function parameters, according to some aspects of the present disclosure and is a variation of that depicted in FIG. 5.

FIGS. 11A, 11B and 11C depict an example of a subject utilizing a system according to the present disclosure to assess the subject's pulmonary lung function where the system is a variation of that depicted in FIGS. 6A and 6B.

FIG. 12 depicts an example application of an embodiment of a subject method for pulmonary function testing of a subject according to the present disclosure and is a variation of that shown in FIG. 7.

### DETAILED DESCRIPTION

Aspects of the present invention include systems comprising a first depth-sensing camera configured to generate depth-sensing images of a subject; and a processor comprising memory operably coupled to the processor, wherein the memory comprises instructions stored thereon, which, when executed by the processor, cause the processor to: identify, based on a reference image received from the depth-sensing camera, reference locations of certain features of interest on the subject; determine a chest region of interest comprising a chest area of the subject, based on the location of the features of interest; receive a plurality of images of the subject from the depth-sensing camera while the subject performs specified breathing maneuvers; generate a three-dimensional representation of the chest region of interest based on the plurality of images of the subject; compute changes in the volume of the chest region of interest based on the three-dimensional representation of the chest; plot the changes in volume of the chest region of interest on a graph, wherein the graph comprises volume of the chest region of interest over time and wherein certain chest movements are labeled on the graph; filter the data on the graph using one or more specified filters; generate a flow volume curve based at least in part on rescaling the filtered graph and computing a gradient of the rescaled graph; compute lung function parameters based at least in part on the flow volume curve; and determine potential clinical interpretations of the pulmonary function of the subject based at least in part on the computed lung function parameters; and an operable connection between the depth-sensing camera and the processor. Also provided are methods of performing pulmonary function testing of a subject.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the system and method may be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

As summarized above, the present disclosure provides methods and systems for pulmonary function testing of a subject. By pulmonary function testing, it is meant assessing how well a subject's lungs are working and providing potential insights into a subject's underlying lung condition, for use in diagnosis and/or treatment of the subject. As such, methods and systems are provided for assessing pulmonary function of a subject using established or standardized metrics in the field, such as forced expiratory volume for one second (FEV1), forced expiratory volume (FEV) or forced vital capacity (FVC) or less established or less standardized metrics such as forced expiratory volume for six seconds (FEV6) or novel metrics that are not yet created or established in the field. Assessing pulmonary function of a subject may also comprise evaluating how effectively a subject performed a prescribed breathing maneuver. In addition, assessing pulmonary function of a subject may also comprise presenting potential interpretations of a subject's pulmonary function as well as potential clinical insights into the subject for diagnosis and/or treatment of the subject. The subject is generally a human subject and may be male or female and of any body type or composition. While the subject may be of any age, in some instances, the subject is not an adult, such as a toddler, juvenile, child, etc. While the subject may be of any body type or body size, in some instances, the subject does not exhibit normal body mass index, such as an underweight, overweight or obese subject.

### Methods for Pulmonary Function Testing

Aspects of the present disclosure include methods for pulmonary function testing. In particular, the present disclosure includes methods for pulmonary function testing of a subject, wherein the method comprises: identifying, based on a reference image received from a depth-sensing camera, reference locations of certain features of interest on a subject; determining a chest region of interest comprising a chest area of the subject, based on the location of the features of interest; receiving a plurality of images of the subject from the depth-sensing camera while the subject performs specified breathing maneuvers; generating a three-dimensional representation of the chest region of interest based on the plurality of images of the subject; computing changes in the volume of the chest region of interest based on the three-dimensional representation of the chest; plotting the changes in volume of the chest region of interest on a graph, wherein the graph comprises volume of the chest region of interest over time and wherein certain chest movements are labeled on the graph; filtering the data on the graph using one or more specified filters; generate a flow volume curve based at least in part on rescaling the filtered graph and computing a gradient of the rescaled graph; computing lung function parameters based at least in part on the flow volume curve; and determining potential clinical interpretations of the pulmonary function of the subject based at least in part on the computed lung function parameters.

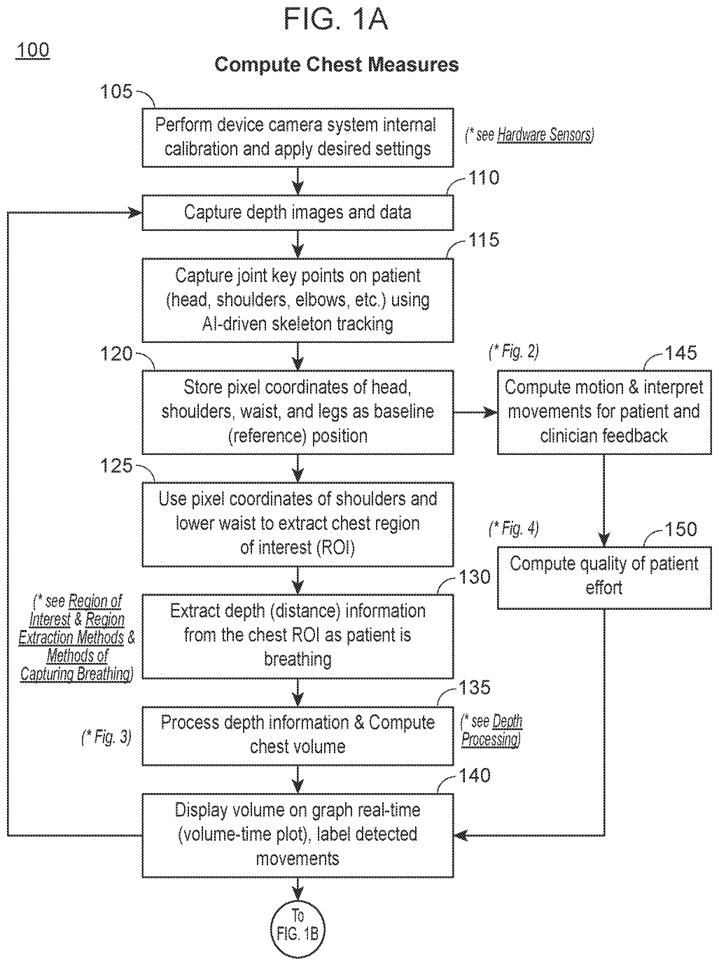

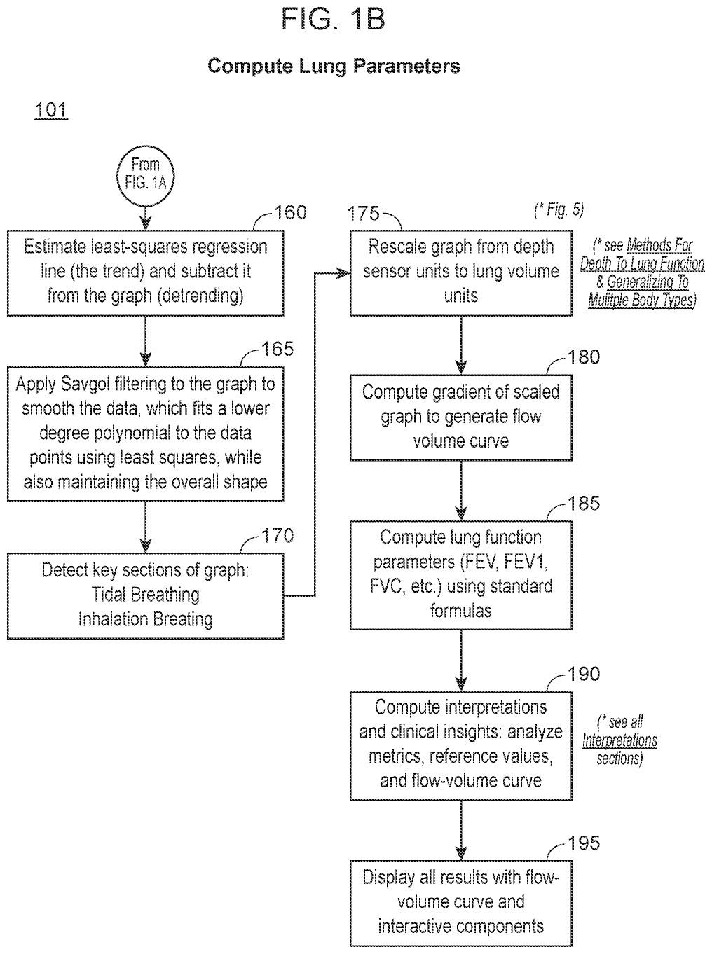

FIGS. 1A and 1B illustrate a flow diagram for pulmonary function testing of a subject according to some aspects of the present disclosure. FIG. 1A illustrates a flow diagram for pulmonary function testing according to some aspects of the present disclosure pertaining to computing chest measures of a subject 100. At block 105 of computing chest measures 100, a depth-sensing camera is initialized. For example, initializing the depth-sensing camera may comprise having the camera conduct an internal calibration, such as an automatic start up routine, including initial checks. The initial calibration of the camera may comprise, in some cases, a standard, automatic initialization routine for the camera device. Block 105 may further comprise applying desired settings to the camera. Applying desired settings to the camera means adjusting any available configurable aspect of the camera (e.g., field of view, focus, etc.) as appropriate in order for the camera to capture depth-sensing images of the subject for use in the methods described herein. In some cases, applying desired settings to the camera may entail, for example, adjusting the field of view of the camera as needed to encompass the subject or certain features of a subject, such as capturing the chest, abdomen or shoulders of the subject in adequate detail; or adjusting the focus settings of the camera to sufficiently distinguish features of a subject from background images that are not relevant to the subject's pulmonary function. In some cases, adjusting camera settings in block 105 may comprise adjusting flash settings or adjusting light-level based settings. In some cases, adjusting camera settings in block 105 may comprise adjusting image resolution settings.

Depth sensing cameras of interest, such as those described above, may be any commercially available, off the shelf, depth-sensing camera, or equivalents thereof. For example, in some cases, depth sensing cameras may be those manufactured by Intel, such as the RealSense Depth Camera models, or those manufactured by ZED.

In some cases, embodiments of the present invention employ more than one depth sensing camera. When more than one depth sensing camera is employed, each of the cameras may be oriented to capture different features of the subject. For example, a first camera may be configured to capture images of the subject's shoulder's and chest, and a second camera may be configured to capture images of the subject's chest and abdomen. In other cases, multiple cameras may be oriented to capture different views of the same features of the subject. For example, a first camera may be configured to capture images of a subject's chest from a front view, and a second camera may be configured to capture images of a subject's chest from a side view. In still other cases, multiple cameras may be oriented to capture images of the same features of the subject at different levels of zoom. For example, a first camera may be configured to capture images of the subject's entire body, and a second camera may be configured to capture images of the subject's chest, exclusively. Any combination of different configurations of multiple cameras may be employed. In addition, embodiments may comprise more than two depth sensing cameras.

Depth-sensing cameras used in embodiments of the present invention may utilize different depth sensing techniques. For example, in some cases, methods according to the present invention may utilize a multi-sensor system, such as one or more depth-sensing cameras, capable of making use of stereo, such as a stereo effect of images captured from different cameras, or time of flight depth sensors or structured light, in each case to determine depth-related information of the captured images. In some cases, methods further utilize temperature sensors to obtain temperature data about the subject or the conditions under which pulmonary testing is taking place. In other cases, methods further comprise using non-depth sensing cameras to acquire images of the subject.

At block 110, one or more images of the subject are captured by one or more depth-sensing cameras. Images captured at block 110 comprise a reference image for use establishing baseline characteristics of the subject. By baseline characteristics, it is meant a characteristic or collection of characteristics (e.g., location of the subject's chest or shoulders, etc.) about the subject. Such aspects of a reference image are recorded such that they can used as a basis of comparison against other images of or data regarding the subject. In some cases, the reference image may be a single image taken by a single depth-sensing camera. In other cases, the reference image may be a combination of more than one images taken by one or more depth-sensing cameras. In certain embodiments, block 110 may further comprise collecting data regarding the subject in addition to a reference image from depth-sensing camera. For example, data regarding the subject's body temperature may be detected by a temperature sensor or non-depth-sensing images may be collected by a camera.

At block 115, one or more key points on the subject are identified for reference. By key points on the subject, it is meant that certain features of interest of the subject are identified. By identified, it is meant that the region of interest in the reference image is associated with a feature of the subject and the location of the region of interest is recorded as being associated with such feature. Any convenient features of interest of the subject may be identified. For example, in embodiments, certain features of interest may comprise a subject's head, shoulders, elbows, chest, abdomen, etc.

Any convenient technique may be used for recognizing key points in an image of a subject. In some cases, identifying key points on the subject may entail utilizing artificial intelligence-based techniques for recognizing features of the subject. Such artificial intelligence-based techniques may comprise any convenient image processing or recognition algorithms, such as those comprising deep learning methods, convolutional neural networks, and the like. In some cases, a reference image undergoes image processing prior to applying image recognition to identify certain key points of the subject. In some cases, a reference “skeleton” model—i.e., a stick model of a partial skeleton of a subject—is superimposed onto the reference image. The skeleton model may be fitted to the reference image based on identifying certain key points of the subject, for example, neck, shoulders, elbows, hips and/or knees of a subject.

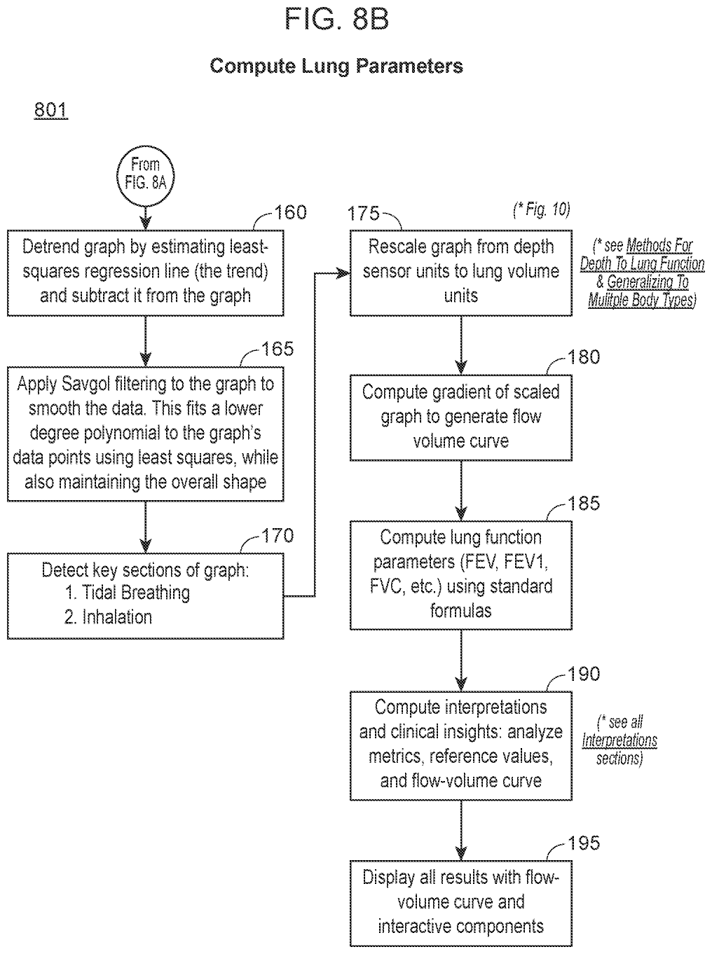

In other embodiments, at block 115, the process proceeds to block 142 as depicted alternative embodiment of flow diagram 800 in FIG. 8A. At block 142 seen in alternative embodiment of flow diagram 800 in FIG. 8A, various clinical metrics are obtained and stored in the system. Relevant clinic metrics may include metrics regarding the subject on which the present invention is applied to ascertain lung function, such as, for example, subject age, subject height, subject body-mass-index (BMI), subject gender and/or subject weight or other metrics applicable to the present techniques. In some cases, such metrics may be obtained from the subject's electronic medical records (EMR) or another applicable cloud-based storage technique, or, in other cases, may be measured and subsequently stored in the subject's electronic medical records or another applicable cloud-based storage technique. In some cases, an algorithm is selected and/or applied to estimate other aspects of the subject. For example, an algorithm may be selected and/or applied to estimate the subject's shoulder width or sitting height. Such estimated measurements may be utilized in connection with refining subsequent estimates of subject lung function.

At block 120, the locations of certain features of interest are stored. By storing certain locations, it is meant that an identifier for the feature of interest associated with, for example, pixel coordinates in the reference image are recorded. In some cases, the pixel coordinates may be three-dimensional pixel coordinates. By stored, it is meant any convenient means of recording information associating a feature of the subject its location in a reference image, such as writing this information into a non-volatile memory, such that the information is present and accessible at a later time. Such locations in the reference image form reference points against which the positions of the subject in subsequent images, such as images of the subject performing specified breathing maneuvers can be compared. As such, locations in the reference image comprise a baseline set of information about the subject.

At block 125, which under certain circumstances is performed after block 120, a chest region of interest of the subject is identified. In embodiments, a chest region of interest may be identified based on the certain features of interest identified in block 120. By identifying a chest region of interest, it is meant the location of a chest region of the subject is identified with reference to the reference image. In some cases, the chest region of interest is located based on pixel coordinates of certain features of interest of the subject. In certain embodiments, the location of the chest region of interest of the subject may be identified based on locations of the subject's right and left shoulders and the subject's waist, such as the subject's lower waist. That is, the location of a subject's chest region of interest may be defined to be an area or a volume contained within certain boundaries specified by features of interest, such as those identified in block 120. The location of the chest region of interest may be identified using pixel coordinates, such as three-dimensional pixel coordinates, by reference to the reference image of the subject.

In some cases, other regions of interest are identified at block 125, in addition to a chest region of interest. In some cases, such as when more than one depth sensing camera or a non-depth sensing camera is used, the chest region of interest may comprise a front chest region of interest and a back chest region of interest, meaning regions of interest determined based on a view of the chest from the front of the subject and a view of the chest from the back of the subject. Other potential regions of interest may comprise a throat region, a neck region, a shoulder region, facial, mouth or lip regions or other accessory muscles. Such additional regions of interest may be used to directly or indirectly measure pulmonary function. In other cases, such additional regions of interest may be used to help identify or confirm aspects of pulmonary function measured based on the chest region of interest.

At block 130, depth information about the chest region of interest of the subject is determined. By depth information, it is meant distance information, such as distance measurements or determinations based on a depth-sensing camera, such as distances between the depth-sensing camera and the chest region of interest on the subject. Such depth information is determined at block 130 while the patient is breathing. In some cases, the patient performs specified breathing maneuvers, such as, for example, standard breathing maneuvers used in the context of traditional spirometry assessment. For example, in some cases, the subject may take a deep breath in, hold the breath for a specified period of time, and exhale as hard as the subject can, or some combination thereof or other possible breathing maneuvers such as inhaling as deeply as the subject can. In other cases, the subject may perform spontaneous breathing. In embodiments, depth or distance information is determined while the subject is breathing by collecting a series of images of the subject with the depth-sensing camera while the subject is breathing. Depth measurements may be determined for each of such a plurality of images or a selection thereof, as appropriate, to obtain sufficient information to assess the subject's breathing. In some cases, the plurality of images is analyzed to determine the image in which the chest region of interest corresponds to a maximum distance from the distance in the reference image (or other applicable metric indicating a difference against the reference image).

In some cases, in block 130, depth or distance information is determined for regions of interest other than the chest region of interest, such as those discussed above in connection with block 125, in each case, while the subject is breathing. For example, in some cases, depth or distance information is determined for a throat region of interest, a neck region of interest, a shoulder region of interest, facial, mouth or lip regions of interest or regions of interest comprising accessory muscles.

In different embodiments, in connection with both blocks 125 and 130, various region extraction methods are used to identify regions of interest, such as a chest region of interest. In some cases, a single-region-region-extraction technique is used. By single-region-region-extraction technique, it is meant, for example, an algorithm that takes as input, exclusively, a single specific region of the subject for certain computations. For example, a single-region-region-extraction technique may take as input exclusively a region associated with the subject's chest, or the subject's shoulders, or the subject's neck, or the like. In other cases, a multi-region-region-extraction technique is used. By multi-region-region-extraction technique, it is meant, for example, an algorithm that takes as input more than one region of a subject for certain computations. For example, a multi-region-region-extraction technique may take as input two or more regions of the subject, such as, for example, the subject's chest and neck. In some cases, in block 130, when a multi-region-region extraction technique is applied, depth measurements are computed for more than one region of the subject, such as, for example, the subject's chest and neck or the subjects, chest, neck and shoulders, or other combinations of identified regions of the subject.

At block 135, depth information computed at block 130 is processed and a volume of the subject's chest is computed. Any convenient technique for computing the subject's chest volume based on certain regions of interest identified in the depth sensing images, such as those obtained at blocks 110 and 130, may be applied. In some cases, the volume of the subject's chest is of interest because changes in volume of the subject's chest during breathing are associated with the volume of air inhaled and exhaled by the subject, and furthermore rates of change of the volume of a subject's chest are associated with rates of breathing, such as rate of inhale or exhale, by the subject.

By processing depth information, it is meant analyzing depth information of one or more images of the subject by applying certain filtering techniques to the images of the subject taken while the subject is breathing. For example, filters or other processing techniques that may be applied include, but are not limited to, a decimation filter, spatial filtering, temporal filtering, hole/loss of data filling, or image sharpening, histogram equalization and/or de-blurring. Filtering and other processing techniques may be applied individually or in combination to images of the subject.

In some cases, the subject's chest volume is computed while the subject is breathing by ascertaining, primarily, the volume of the subject's chest region of interest by: (1) capturing depth information (of the chest region of interest (or, in other cases, of any of the possible region of interests)) using one or more depth-sensing cameras, as discussed in connection with block 130; (2) converting depth pixel values and coordinates in the obtained images into three-dimensional point-clouds; (3) generating mesh triangulations from point clouds using algorithms such as Delaunay and Marching Cubes, each of which is known in the field; (4) applying surface textures to such generated mesh for display and further analysis; and (5) with the generated meshes, voxel volumes can be generated using integration and summed to compute a final chest volume of the subject.

In other cases, the subject's chest volume is computed while the subject is breathing based on an image processing based technique focused on the displacement of certain regions of interest on the subject, such as, for example, the subject's neck and/or shoulders. Such technique may comprise the following steps: (1) extracting multiple (for example, 10 to 20 or more) feature points along the subject's shoulder and neck in the images; such feature points can be identified and extracted from images using any of the various feature extractor algorithms known in the field (for example, SIFT); (2) such feature points are tracked, frame by frame, i.e., among the plurality of images showing the subject breathing; such feature points can be tracked using any of the various tracking algorithms that are known in the field (for example, Kanade-Lucas-Tomasi (KLT)), which functions by measuring the dissimilarity of transformation parameters for each key point across frames (i.e., images); and (3) displacement of key points across each frame of the plurality of images captured from the depth-sensing cameras is computed. Such displacement values may be further analyzed to reflect changes in the subject's chest volume.

In still other cases, the subject's chest volume is computed while the subject is breathing based on a deep learning based technique focused on the displacement of certain regions of interest on the subject, such as, for example, the subject's neck and/or shoulder. Such technique may comprise the following steps: (1) extracting key points in the images using artificial intelligence-driven skeleton (joint) tracking; (2) tracking key points using artificial intelligence-driven skeleton (joint) tracking, such as, for example, tracking changes of location in such key points across some or all of the plurality of images taken while the subject is breathing; and (3) computing displacement of key points across frames of the plurality of images captured from the depth-sensing cameras. Such displacement values may be further analyzed to reflect changes in the subject's chest volume.

At block 140, information regarding the volume of the chest region of interest of the subject is displayed on, for example, an output device such as a monitor, device screen or the like. In embodiments, volume information is displayed in the form of a graph, such as a graph of chest volume over time, representing chest volume calculated for the plurality of images taken using a depth-sensing camera while the subject is, for example, performing breathing exercises or breathing spontaneously. In embodiments, the graph of chest volume over time illustrates changes in chest volume, where changes in chest volume are associated with the volume of air taken in by the subject during breathing. Similarly, the shape of the graph of the chest volume over time is associated with certain breathing rates, such as the rate of inhalation or exhalation. A graph of chest volume over time may be referred to as a volume-time plot.

In embodiments, at block 140, certain detected movements may be labeled. For example, when changes in chest volume over time correspond to inhalation or exhalation movements, such regions of the graph may be labeled as such. In other examples, when the subject is performing scripted breathing maneuvers, components of the breathing maneuvers may be labeled on the graph, such as, for example, a period of time when the subject exhales forcefully or as hard as the subject can. Any convenient form of labeling areas of the graph may be used, such as color coding sections of the graph or overlaying text labels onto the graph.

In some cases, the chest volume of the subject may be determined in real time or near real time based on images of the subject collected in real time, or near real time, from one or more depth-sensing cameras and, in some cases, one or more non-depth sensing cameras. When chest volume is computed in real time, the displayed graph may correspondingly be updated in real time such that the graph displays real time values of the subject's chest volume—i.e., real time results of the volume of air inhaled and exhaled by the subject over time. Similarly, labels of certain events, such as certain breathing maneuvers, may be added to the graph in near real time, as they are identified.

At block 145, motion by the subject is identified based on a plurality of images obtained from the depth-sensing camera. In particular, the subject's motion in such plurality of images is determined by comparing observed motion against the reference image and reference locations of certain features of interest of the subject. That is, location information of certain features of interest of the subject may be computed based on the plurality of images and such location information may be compared against the reference image to identify certain movements of the subject. Once the existence of certain movements of the subject are identified through computation of changes in location information, information about such movements may be interpreted in embodiments of the present disclosure. By interpreting movement, it is meant that, in some cases, movements other than breathing are identified. In other cases, movements associated with breathing may be identified. In embodiments, upon detection and identification, interpretations of movements of the subject may be output to a user, such as the subject or a clinician treating the subject. In some cases, the form of output may be to display additional information on the graph of chest volume over time described above in connection with block 140. Identification and interpretation of the subject's movements is useful in ascertaining whether the subject is performing breathing maneuvers as described. In particular, at block 150, the quality of the subject's effort is computed. By quality of the subject's effort, it is meant that, based on results obtained through analysis of depth-sensing images, as described above, it is determined, for example, that the subject is not performing breathing maneuvers as directed or that the subject is not performing the breathing maneuvers to the extent required for an accurate assessment of pulmonary function. Such determinations, and the chest volumes and times associated therewith, may also be labeled on the graph of chest volume versus time described above in connection with block 140.

FIG. 1B illustrates a flow diagram for pulmonary function testing according to some aspects of the present disclosure pertaining to computing lung parameters of a subject 101. At block 160, which follows execution of block 140 in FIG. 1A, the data of the graph described above in connection with block 140 in FIG. 1A is filtered. Specifically, in embodiments, at block 160, trend information summarizing certain data of the graph is estimated using any convenient method of estimating a trend in a collection of data. For example, in some cases, a least squares method is used to estimate a regression line for certain of the chest volume over time data that is displayed on the graph. Once trend information about certain data presented on the graph is estimated, the trend information is used to detrend the graph. Any convenient technique for detrending the graph may be used. For example, in some cases, after a regression line is identified, it is subtracted from the applicable data presented on the graph in order to detrend the graph.

At block 165, the graph data is smoothed using additional filtering techniques. Any convenient graph-smoothing technique may be applied. For example, Savgol filtering may be applied to the graph in order to smooth the data. Such filtering entails fitting a lower degree polynomial to the data points using least squares, which is a known curve fitting technique in the field, while also maintaining the shape of the graph. By lower degree polynomial, it is meant a polynomial of degree one or more, two or more, three or more, five or more, six or more, seven or more, eight or more or nine or more, such as a polynomial of degree three. Maintaining the overall shape of the graph may be achieved by selecting a polynomial of appropriate degree. Upon filtering the graph in block 165, the resulting graph more accurately reflects changes in the subject's chest volume, and therefore volume of air entering and exiting the subject, because after filtering, fewer aspects of the graph reflect noise, such as measurement errors. For purposes of evaluating pulmonary function, it is important that the graph accurately reflect volumes of air exchanged by the subject's lungs, which is why filtering is applied to the graph, such as in block 165, as well as block 160. Similarly, while it is important that filtering be applied in certain embodiments, filtering is nonetheless applied in such a manner that the overall shape—and therefore data regarding changes in air volume inhaled and exhaled by the subject—is preserved.

At block 170, key sections of the graph are identified. Key sections of the graph may comprise any section of the graph that is of interest for a particular pulmonary function test. For example, in embodiments, sections of the graph that are associated with tidal breathing or inhalation or exhalation or passive breathing or other breathing may be identified. Any convenient technique may be applied for identifying such key sections of the graph. For example, in some cases, image recognition algorithms, such as, for example, but not limited to, signal processing algorithms or artificial intelligence image recognition algorithms, may be applied to identify particular characteristics of the graph when certain breathing maneuvers are performed. That is, in some cases, certain breathing maneuvers are expected to have characteristic shapes on the graph, which shapes may be leveraged in order to identify key sections of the graph. In other cases, certain criteria about characteristics of the graph may be applied to identify key sections. In other cases, key sections of the graph may be identified based at least in part on correlating the time at which the subject is instructed to perform a specific breathing maneuver and corresponding changes in the volume of the chest region of interest at such specified times.

At block 175, the graph is rescaled from depth sensor units to lung volume units. By rescaling from depth sensor units, it is meant converting the data presented on the graph from units corresponding to a volume—or changes in volume—of the subject's chest region of interest into units corresponding to the subject's lung volume. Any convenient technique for rescaling units of a graph may be applied at block 175.

In some embodiments, rescaling from depth sensor units to lung volume units comprises computing calibration curves. In embodiments, computing calibration curves comprises: (1) a plurality of lung volume curves (e.g., two or more, three or more, four or more, five or more) are generated for a subject using a traditional spirometer, where the lung volume curves reflect data comprising lung volume and time measurements, such as a lung volume versus time graph or other similar, such as other graphs used in the field; (2) a polynomial (such as a second, third, fourth or fifth or greater degree polynomial) is fitted to the set of lung volume curves to generate a fitted model; and (3) a calibration curve (i.e., a graph of lung function) is applied to a curve generated by a sensor (i.e., a curve generated by, for example, the depth-sensing camera discussed above).

In other embodiments, rescaling from depth sensor units to lung volume units comprises applying scaling factors generated by, for example, linear regression. In embodiments, applying scaling factors generated by linear regression comprises: (1) generating a linear model for a subject that minimizes the difference between the curve computed from a device, such as a depth-sensing camera, as described above, to a spirometer; (2) select a subset of curves generated by a spirometer as a training set; and (3) using data points from a spirometer and data points from a device, such as a depth-sensing camera, to conduct linear regression to learn the scaling factors that translate the units of the device to spirometer units.

In embodiments, at block 175, rescaling from depth sensor units to lung volume units comprises generalizing to multiple body types. For computations of the volume of a chest region of interest of a subject based on depth-sensing images to be accurate, such computations must be applicable to multiple different body types, such as different shapes of bodies. Any convenient approach may be applied to generalize the above described techniques for rescaling from depth sensor units to lung volume units to multiple body times.

In some embodiments, a clustering technique is applied. A clustering-based technique for generalizing to multiple body types may be based on accessing a large dataset (meaning a dataset comprising a plurality of different measurements from a plurality of different body types). With such a large data set, different shapes, ages, genders, and other characteristics of the subjects can be clustered into different groups using, for example, machine learning based clustering methods. Scaling factors for use in rescaling from depth sensor units to lung volume units may be computed for each such cluster. Since different scaling factors may apply to different groups of subject characteristics, similar patient populations will have similar scaling factors.

In other embodiments, a deep-learning based technique is applied. A deep learning-based technique for generalizing to multiple body types may similarly be based on accessing a large dataset (again, meaning a dataset comprising a plurality of different measurements from a plurality of different body types). With such a large data set, a custom neural network architecture is created to learn the scaling factors based on the different shapes, ages, genders, etc. reflected in the data set. As described above, different scaling factors used to rescale from depth sensor units to lung volume units may apply for different body types. In a deep-learning based approach, different scaling factors are computed based on application of a neural network architecture to the data set.

At block 180, a flow volume curve is generated based on computing a gradient of the graph, after the graph has been rescaled to lung volume units at block 175. In embodiments, a flow volume curve may comprise a plot of the subject's lung volume with the rate of change of the subject's lung volume. Gradient refers to a rate of change, in this case with respect to the subject's lung volume—and therefore volume of air inhaled or exhaled—over time. That is, at block 180, one or more rates of change of the subject's lung volume is computed and plotted with respect to the subject's lung volume in order to generate a flow volume curve reflecting the subject's performance of breathing maneuvers. In some cases, flow volume curves are generated in a format that is commonplace and/or standardized in the field; in some cases, flow volume curves are generated that are not commonplace and/or standardized in the field.

At block 185, lung function parameters are computed based on the flow volume curve generated at block 180. In some cases, standard lung function parameters are generated according to standard techniques for generating such parameters based on a typical format flow volume curve, as is known in the field. Any convenient lung function parameter capable of being generated from a flow volume curve may be generated at block 185. In some cases, standard lung function parameters such as forced exhalatory volume (FEV), or forced exhalatory volume in one second (FEV1), or forced vital capacity (FVC) may be computed. In other cases, less common or novel lung function parameters may be generated based on the flow volume curve, such as forced exhalatory volume in six seconds (FEV6). Standard formulas, known in the field, may be applied to generate lung function parameters based on the flow volume curve generated at block 180.

At block 190, interpretations and clinical insights regarding the subject's pulmonary lung function are computed. Interpretations and clinical insights may be computed based on any available information about the subject, such as, for example, lung function parameters computed at block 185 as well as the flow volume curve computed at block 180, and the graph computed at block 140. Interpretations and clinical insights may further incorporate other information about a subject, such as the subject's medical history or other clinical characteristics of the subject. Embodiments may analyze metrics, reference values, and one or more flow volume curves, in each case in a quantitative and/or qualitative manner, in order to compute interpretations and clinical insights regarding the subject's pulmonary function. The computed interpretations and clinical insights may be quantitative and/or qualitative in nature. Such interpretations and clinical insights about the subject may be presented to a user, such as the subject, in any convenient manner, such as by displaying such information on a display device, such as a monitor or the like.

Embodiments are configured to choose efforts (i.e., breathing maneuvers performed by the subject) to interpret at block 190 even if the breathing maneuvers and/or the associated interpretations and clinical insights are not standardized, such as, not standards of the American Thoracic Society (ATS). In such cases, embodiments are configured such that at block 190, an algorithm may be applied to select and analyze certain spirometry efforts that still provide information of interest, even if such spirometry efforts and associated insights and clinical interpretations are not considered ATS standards or ATS qualified.

By qualitative interpretations and clinical insights, it is meant interpreting pulmonary function of the subject using qualitative features of the data collected or computed, such as, for example, the shape, sharpness, peak-ness of a graph of collected and/or computed data. In embodiments, certain shape metrics are computed using imaging processing based first and second order feature analysis and computer-vision based image extraction.

By quantitative interpretations and clinical insights, it is meant interpreting pulmonary function of the subject using quantitative metrics of the data collected or computed, such as, for example, of a graph, such as, for example, taking account of all the data points in the flow-volume curves of the subject's breathing maneuvers. In other embodiments, quantitative interpretations and clinical insights may entail using discrete values wherein many of the standard and common lung function parameters are computed, such as, for example, FEV1, FEV, FVC, etc. In some cases, new and/or not yet standardized, metrics, such as forced expiratory volume in six seconds (FEV6), may be computed and included in the interpretations and clinical insights, including as inputs into algorithms configured to generate interpretations and clinical insights of the subject's pulmonary function based on lung function parameters. In other cases, certain machine learning algorithms and/or signal processing algorithms are used for analysis for generating interpretations and clinical insights. In embodiments, reference equations are used in connection with computing quantitative interpretations and clinical insights at block 190. In some cases, reference equations will be used and included in an algorithm configured to compute predicted values based on certain features of a subject, such as gender, race, age, height, etc.

At block 195, all results related to the pulmonary function of the subject are displayed along with a flow volume curve that includes interactive components. Any convenient form of display may be applied, for example, a display centered around a flow volume curve representing the subject's pulmonary function. In embodiments, the display may be a user friendly, visual graph configured for use by a physician, for example, a pulmonologist. In embodiments, the display comprises the graphs, such as those graphs described above, analysis, and interpretations in an easy-to-read manner.

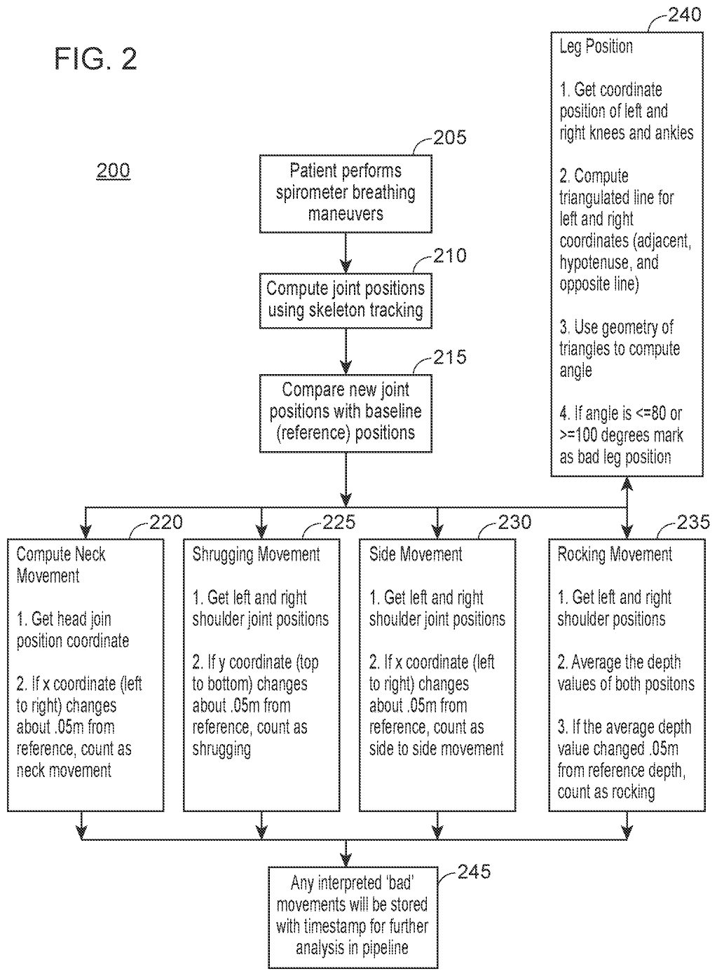

FIG. 2 depicts a flow diagram 200 for computing motion and interpreting movements of the subject according to some aspects of the present disclosure, such as for use in providing feedback to the subject or to a clinician. In embodiments, the flow diagram 200 for computing motion and interpreting movements of the subject represents additional detail of steps that occur in connection with block 145 in FIG. 1A. At block 205 of computing motion and interpreting movements of the subject 200, the subject performs specified breathing maneuvers, such as, for example, spirometry breathing maneuvers. Any convenient breathing maneuver may be specified in connection with block 205 and may be selected based on the pulmonary function parameters and interpretations and insights of interest to the subject. Breathing maneuvers such as those discussed above in connection with block 120 of FIG. 1A may be performed. Images of the subject while performing breathing maneuvers may be captured, such as images captured by depth sensing cameras while the subject is breathing, as discussed above in connection with FIG. 1A.

At block 210, key points on the subject, such as certain joint positions, are identified and tracked. Such key points may be identified and tracked based on a plurality of images collected from a depth-sensing camera at block 205. Any convenient technique for identifying key points and certain joint positions on the subject may be applied. For example, skeleton tracking techniques may be used for identifying and tracking key points. In embodiments, any technique for key point identification and tracking discussed above in connection with block 115 or block 120 of FIG. 1A above may be applied.

At block 215, the locations of key points identified and tracked at block 210 are compared against locations of key points identified in a reference image of the subject, such as the locations of key points of the subject in a reference image discussed above in connection with block 120 of FIG. 1A, as well as blocks 110, 115 and 125 of FIG. 1A. Such comparison against one or more reference images may indicate movement by the subject, such as, for example, chest movement associated with breathing or moving an arm. That is, in some cases, movements identified at block 215 by comparison against a reference location are associated with breathing movements and in other cases such movements are associated with movements other than breathing movements.

At block 220, changes in location of key points on a subject computed at block 215 are analyzed to determine whether such movements may be caused by neck movement by the subject. In embodiments, a movement is identified and tagged as neck movement by: (1) computing a head joint position coordinate, based on the locations of key points of the subject identified in block 215; and (2) if the x coordinate (i.e., the location of the head joint in a left to right, relative to the subject, frame of reference) changes by a specified amount from the reference location of the same head joint location, the movement is tagged and identified as neck movement. Any convenient specified amount of movement from the reference location of the head joint of the subject may be applied, such as, for example, an amount greater than 0.01 m or 0.02 m or 0.05 m or an amount greater than 0.1 m.

At block 225, changes in location of key points on a subject computed at block 215 are analyzed to determine whether such movements may be caused by a shrugging movement by the subject. In embodiments, a movement is identified and tagged as a shrugging movement by: (1) computing left and right shoulder joint positions of the subject in the plurality of images received in connection with block 205 as well as the reference image; and (2) if the y coordinates (i.e., the locations of the left and right shoulder positions in a top to bottom, relative to the subject, frame of reference) changes by a specified amount from the reference locations of the same left and right shoulder joint positions, the movement is tagged and identified as shrugging. Any convenient specified amount of movement from the reference location of the left and right shoulders of the subject may be applied, such as, for example, an amount greater than 0.01 m or 0.02 m or 0.05 m or an amount greater than 0.1 m.

At block 230, changes in location of key points on a subject computed at block 215 are analyzed to determine whether such movements may be caused by a side movement by the subject. In embodiments, a movement is identified and tagged as a side movement by: (1) computing left and right shoulder joint positions of the subject in the plurality of images received in connection with block 205 as well as the reference image; and (2) if the x coordinates (i.e., the locations of the left and right shoulder positions in a right to left, relative to the subject, frame of reference) changes by a specified amount from the reference locations of the same left and right shoulder joint positions, the movement is tagged and identified as a side movement. Any convenient specified amount of movement from the reference location of the left and right shoulders of the subject may be applied, such as, for example, an amount greater than 0.01 m or 0.02 m or 0.05 m or an amount greater than 0.1 m.

At block 235, changes in location of key points on a subject computed at block 215 are analyzed to determine whether such movements may be caused by rocking movement by the subject. In embodiments, a movement is identified and tagged as a rocking movement by: (1) computing left and right shoulder joint positions of the subject in the plurality of images received in connection with block 205 as well as the reference image; (2) computing the average of the depth values of both positions (i.e., the distance between each key joint position and a depth sensing camera); and (3) if the average depth value of the left and right shoulder positions changes by a specified amount from the reference locations of the same left and right shoulder joint positions, the movement is tagged and identified as a rocking movement. Any convenient specified amount of change in depth from the reference location of the left and right shoulders of the subject may be applied, such as, for example, an amount greater than 0.01 m or 0.02 m or 0.05 m or an amount greater than 0.1 m.

At block 240, changes in location of key points on a subject computed at block 215 are analyzed to determine whether such movements may be caused by changes in leg position by the subject. In embodiments, a movement is identified and tagged as changes in leg position by: (1) computing left and right knee and ankle joint positions of the subject in the plurality of images received in connection with block 205 as well as the reference image; (2) computing a triangulated line for left and right coordinates (adjacent, hypotenuse, and opposite line) related to the subject's leg position; (3) computing relevant angles of leg components using, for example, the geometry of triangles; and (4) if the computed angle exceeds a specified threshold, the movement is tagged and identified as changes in leg position, such as a bad leg position, meaning, for example, an undesirable leg position for purposes of conducting pulmonary function testing. Any convenient specified threshold angle related to leg position of the subject may be applied, such as, for example, if the angle is less than or equal to 80 degrees or 70 degrees or 90 degrees, or if the angle is greater than or equal to 100 degrees or 90 degrees or 110 degrees.

At block 245, motions identified at any of blocks 220, 225, 230, 235 or 240 may be associated with a timestamp of when such movements occurred, and stored, such as stored in a non-volatile memory, for later review by the subject or by a clinician. Any convenient information about the analysis of such movements may also be stored for later review and analysis.

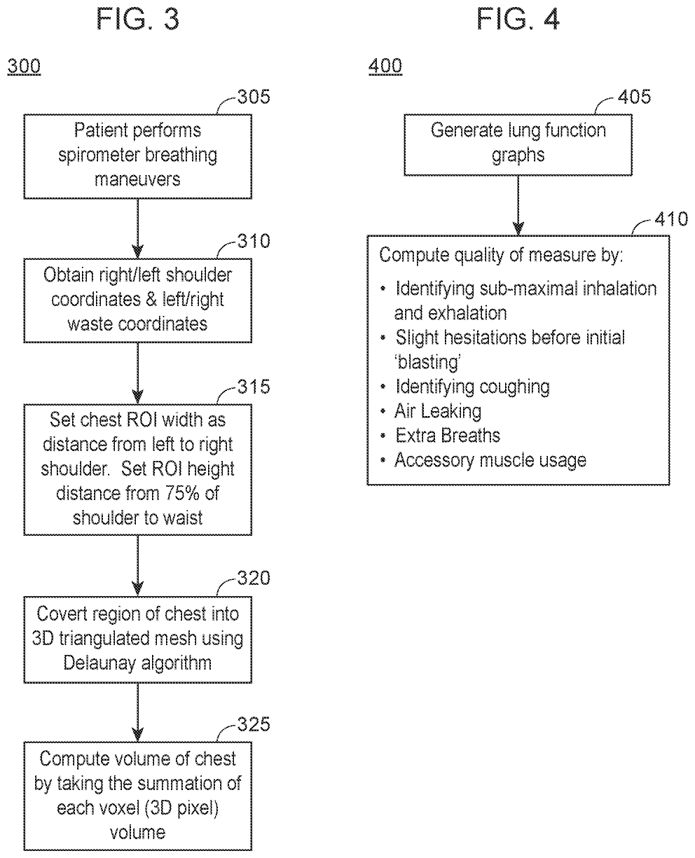

FIG. 3 depicts a flow diagram 300 for processing depth information and computing chest volume of the subject according to some aspects of the present disclosure. In embodiments, the flow diagram 300 for computing motion and interpreting movements of the subject represents additional detail of steps associated with block 135 in FIG. 1A. At block 305 of processing depth information and computing chest volume of the subject 300, the subject performs specified breathing maneuvers, such as, for example, spirometry breathing maneuvers. Any convenient breathing maneuver may be specified in connection with block 305 and may be selected based on the pulmonary function parameters and interpretations and insights of interest to the subject. Breathing maneuvers such as those discussed above in connection with block 120 of FIG. 1A may be performed. Images of the subject breathing while performing breathing maneuvers may be captured, such as images captured by depth sensing cameras while the subject is breathing, such as those discussed above in connection with FIG. 1A.

At block 310, key points on the subject, such as right and left shoulder locations and right and left waist locations, are identified and tracked. Such key points may be identified and tracked based on a plurality of images collected from a depth-sensing camera at block 305. Any convenient technique for identifying key points and certain joint positions on the subject may be applied. For example, skeleton tracking techniques may be used for identifying and tracking key points. In embodiments, any technique for key point identification and tracking discussed above in connection with block 115 or block 120 of FIG. 1A above may be applied.

At block 315, a chest region of interest is set based on the subject's shoulder and waist locations. Any technique for defining a chest region of interest may be applied, such as those discussed above in connection with block 125 of FIG. 1A. In embodiments, a chest region of interest may be set to correspond to a subset of the area defined by the subject's shoulders and waist. For example, the chest region of interest may be set to correspond to the area bound by a width that is the distance from the subject's left to right shoulder and a height that is the distance from a specified percentage of the distance between the subject's shoulders and waist, for example 75% of the distance between the subject's shoulders to waist.

At block 320, the chest region of interest identified in block 315 is converted into a three-dimensional representation of the chest region of interest. A three-dimensional representation of the chest region of interest of the subject may be used in computing chest volumes of the subject. Any convenient technique for converting images, such as images taken by a depth-sensor, into a three-dimensional representation may be applied. For example, the techniques discussed above in connection with block 130 and block 135 of FIG. 1A in connection with computing chest volume may be applied. In some embodiments, a chest region of interest may be converted into a three-dimensional representation by converting the chest region of interest into a three dimensional triangulated mesh. In some cases, the Delaunay algorithm, as is known in the field, is applied to the chest region of interest in order to facilitate converting the chest region of interest into a three-dimensional representation, such as a three-dimensional triangulated mesh.

At block 325, a volume of the chest region of interest by the subject is computed based on the three dimensional representation generated at block 320, discussed above. In some cases, a volume of the chest region of interest is computed by taking the summation of each voxel (i.e., each three dimensional pixel) of the three dimensional representation in order to yield the volume of the subject's chest region of interest.

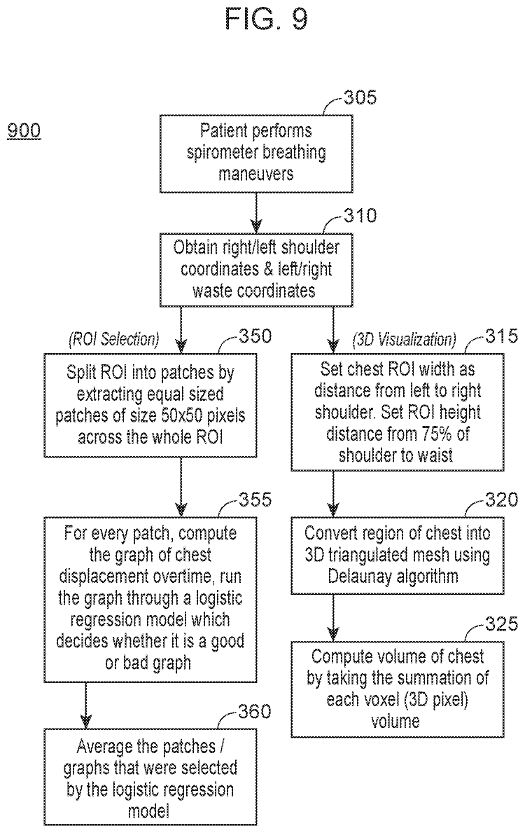

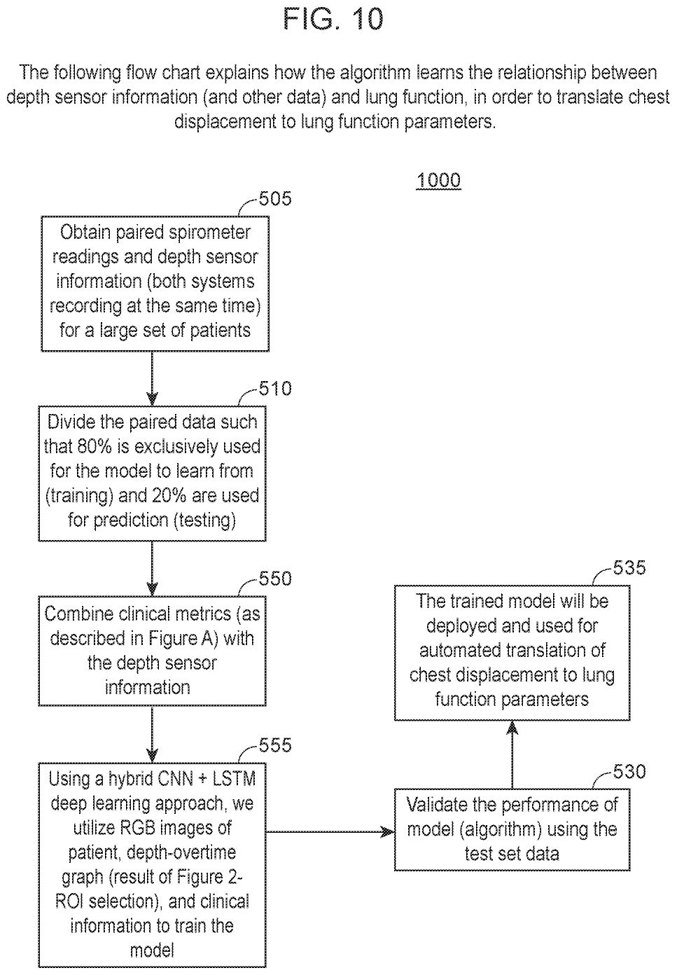

In other embodiments, at block 310, the process proceeds to block 350 in connection with selecting a region of interest (ROI Selection) as depicted in alternative embodiment of flow diagram 900 in FIG. 9. At block 350 a region of interest (ROI) is split into patches by extracting equal sized patches of, for example, size 50×50 pixels across the whole ROI.

At block 355 as depicted in alternative embodiment of flow diagram 900 in FIG. 9, for every patch, the graph of chest displacement overtime is computed, and the resulting graph is run through a logistic regression model, which decides whether it is a good or bad graph. Any convenient logistic regression model may be applied, as such are known in the art. By a good or bad graph, it is meant a determination of whether the graph patch accurately depicts a patch of the ROI.

At block 360 as depicted in alternative embodiment of flow diagram 900 in FIG. 9, the patches/graphs that were selected by the logistic regression model are averaged.

FIG. 4 depicts a flow diagram 400 for computing a quality of effort by the subject when performing breathing maneuvers used to evaluate the pulmonary function of the subject, according to some aspects of the present disclosure. In embodiments, the flow diagram 400 for computing a quality of effort by the subject represents additional detail of steps associated with block 150 in FIG. 1A. At block 405 lung function graphs are generated based on breathing maneuvers performed by the subject, such as those breathing maneuvers discussed above. Lung function graphs may comprise any convenient representation of relevant characteristics of the subject's lung function, such as a graph of chest volume or lung volume over time, as described in greater detail in connection with block 140 of FIG. 1A or block 175 of FIG. 1B. In some cases, lung function graphs may comprise flow volume curves, as discussed above in connection with FIG. 1B.

At block 410, a quality of measure of the subject's quality of effort when performing breathing maneuvers is computed based at least in part on the lung function graphs. In some cases, quality of effort of the subject is computed by: identifying sub-maximal inhalation and exhalation by the subject; identifying slight hesitations before initial ‘blasting’ by the subject; identifying coughing by the subject; identifying air leaking; identifying extra breaths; or identifying accessory muscle usage. In each case such quality of effort signals are identified based at least in part on characteristics exhibited by the lung function graphs.

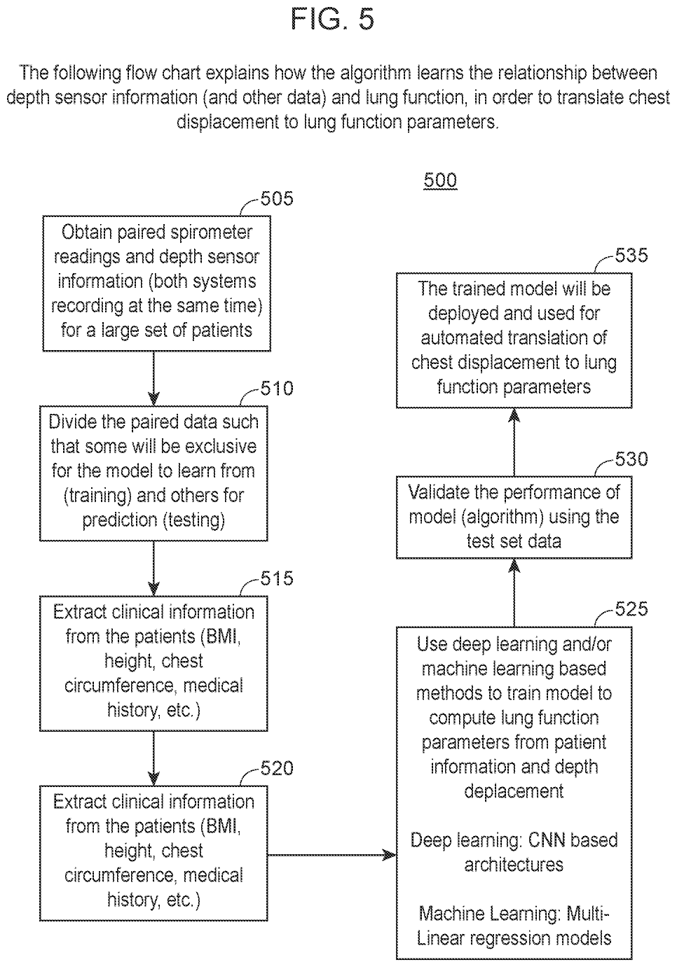

FIG. 5 depicts a flow diagram 500 for computing a relationship between depth sensor information (and other data) and the subject's lung function, in order to translate chest displacement to lung function parameters, according to some aspects of the present disclosure. In embodiments, the flow diagram 500 for computing a relationship between depth sensor information (and other data) and the subject's lung function represents additional detail of steps associated with block 175 in FIG. 1B. In other embodiments, the flow diagram 500 for computing a relationship between depth sensor information (and other data) and the subject's lung function represents steps that may be performed prior to computing lung volume of a particular subject.

At block 505, paired spirometer readings and depth sensor information (both systems recording at the same time) are obtained for a plurality of subjects. The plurality of subjects may consist of two or more subjects, or five or ten or 100 or 1,000 or more subjects. Such subjects may be similarly situated with respect to pulmonary function or may differ. Such subjects may be similarly situated with respect to physical characteristics, such as gender or body type or body size, or may differ. Spirometer readings and depth sensor information may comprise data obtained while the subject is performing prescribed breathing maneuvers or passive breathing. In either case, the spirometer readings may be associated with depth sensor information based on time stamps of when each data point is collected.

At block 510, the paired spirometer and depth sensor data are divided, such as divided into two groups, a first group and a second group, such that data pairs in the first group may be utilized exclusively for training a model to learn to estimate lung function and pairs of data in the second group may be utilized for evaluating the effectiveness of the model by predicting lung function with a known result, i.e., testing the model. Any convenient division of the paired spirometry data may be applied. In some cases, an even number of data pairs are assigned to the first and second groups. In other cases, more data pairs are assigned to one of the two groups. In some cases, the two groups are divided such that each group includes representative examples of different pulmonary lung function of subjects as well as different physical characteristics of subjects, such as gender or body type or body size.

At blocks 515 and 520, certain clinical information regarding the subjects that comprise the first and second groups of subjects is received. Such clinical information may include any convenient and available characteristic of the subjects for use in determining an association between such characteristic and the subject's pulmonary function. In some cases, such clinical information may include one or more of: subject body mass index (BMI), height, chest circumference, certain medical history, such as, for example, a history of lung disease, etc. In embodiments, clinical information about the subjects is associated with the spirometry and depth sensor data collected about each subject.

At block 525, the model is trained to compute lung function parameters from patient information and depth displacement obtained from depth sensors. The model may be training using any convenient technique. In embodiments, the model may be trained using deep learning techniques. When the model is trained using deep learning techniques, the model and/or its training may be based at least in part on convolutional neural network (CNN) architectures. In other embodiments, the model may be trained using machine learning based techniques. When the model is trained using machine learning based techniques, the model and/or its training may be based at least in part on multi-linear regression models. In embodiments, data used to train the model is the first data set defined at block 510, described above.

In other embodiments, at block 510, the process proceeds to block 550 as depicted alternative embodiment of flow diagram 1000 in FIG. 10. At block 550 as depicted alternative embodiment of flow diagram 1000 in FIG. 10, clinical metrics (as described above in connection with block 142 of FIG. 8A) are combined with information obtained from depth sensor (i.e., depth sensor information). Such information may be combined in any convenient manner, such as, for example, combined in any convenient data structure or data base or EMR-type records or cloud-based storage or the like.

At block 555 as depicted alternative embodiment of flow diagram 1000 in FIG. 10, a hybrid convolutional neural network (CNN) and long short-term memory (LSTM) deep learning approach, as such artificial intelligence algorithms are known in the art, is used. RGB images of subject, depth-overtime graph (e.g., the results of ROI selection described above in connection with blocks 350, 355 and 360 of alternative embodiment 900 depicted in FIG. 9), and clinical information may be used to train the model comprising a convolutional neural network (CNN) and long short-term memory (LSTM) deep learning approach.

At block 530, the performance of the model that was trained at block 525 is validated using test data. That is, if the first data set defined at block 510 above was used to train the model at block 525, then the second data set, also defined at block 510 above, is used to validate the model at block 530. Any convenient technique may be used for validating the model. For example, depth sensor and other clinical data about a certain subject in the second data set may be input to the model and the prediction produced by the model may be compared against the spirometer data obtained for the certain subject. When lung function parameters computed based on the spirometer and lung function parameters predicted by the model based on depth sensor data yield identical or near identical results of pulmonary lung function of the subject, then such result supports a finding that the model is capable of predicting accurate results of lung function. When, on the other hand, lung function parameters computed based on the spirometer and lung function parameters predicted by the model based on depth sensor data yield different or incompatible results of pulmonary lung function of the subject, then such result supports a finding that the model may not capable of predicting accurate results of lung function.

At block 535, the model may be deployed and used for automated translation of chest displacement to lung function parameters. That is, the model, after it has been trained, as described in connection with block 525, and validated, as discussed in connection with block 530, the model may be applied to depth sensing images generated based on a subject that is not a member of the first or second groups of existing paired data, as described above in connection with blocks 505 and 510. That is, the model may be applied to predict lung function of subjects for which there is no spirometer data available. In other words, no spirometer or spirometry based data is required to predict lung function parameters of a subject at block 535.

FIG. 8A and FIG. 8B illustrate an alternative flow diagram for conducting pulmonary function testing according to embodiments of the present invention that is a variation of that depicted in FIG. 1A and FIG. 1B. FIG. 8A comprises a flow diagram for computing a chest measures aspect of the invention 800, and FIG. 8B comprises a flow diagram for computing a lung parameters aspect of the present invention 801. Flow diagram for pulmonary function testing according to some aspects of the present disclosure pertaining to computing chest measures of a subject 800 is similar to flow diagram 100 seen in FIG. 1A, and flow diagram 101 depicted in FIG. 1B is similar to flow diagram 101 seen in FIG. 1B, such that descriptions of identical blocks in respective flow diagrams are not separately described. As described above, FIG. 8A includes block 142, described above, which in some cases, in flow diagram 800, follows block 115.

FIG. 9 depicts an alternative flow diagram 900 for processing depth information and computing chest volume of a subject according to some aspects of the present disclosure that is a variation of flow diagram 300 that is depicted in FIG. 3. Flow diagram 900 is similar to flow diagram 300 seen in FIG. 3, such that descriptions of identical blocks in respective flow diagrams are not separately described. As described above, FIG. 9 includes blocks 350, 355 and 360, described above, which in some cases, in flow diagram 900, follows block 310.

FIG. 10 depicts an alternative flow diagram 1000 for computing a relationship between depth sensor information (and other data) and a subject's lung function, in order to translate chest displacement to lung function parameters, according to some aspects of the present disclosure and is a variation of that depicted in FIG. 5. Flow diagram 1000 is similar to flow diagram 500 seen in FIG. 5, such that descriptions of identical blocks in respective flow diagrams are not separately described. As described above, FIG. 10 includes blocks 550 and 555, described above, which in some cases, in flow diagram 1000, follow block 510.

In addition to the aspects of the present disclosure described above, in some cases, embodiments of the methods comprise coaching a subject in connection with assessing pulmonary function of the subject. For example, in some cases, the subject is coached when performing breathing maneuvers, or otherwise, while images of the subject are collected.

In some cases, the subject is coached with respect to movement and position detection. For example, rocking back and forth by the subject may be detected; in some embodiments, an algorithm may be configured to track the movement of the subject across the chest and shoulders in real-time in order to detect if the subject is rocking back and forth. When rocking back and forth is detected and the rocking is determined to be moderate to severe rocking, the algorithm may be configured to alarm and/or warn the subject and/or a clinician. Another example is detecting side movement by the subject; in other embodiments, an algorithm may be configured to track movement of the subject's shoulders and upper chest to detect left-to-right movement by the subject. When such side movement is detected and the movement is determined to be moderate to severe movement, the algorithm may be configured to alarm and/or warn the subject and/or a clinician. Still another example is detecting leg positioning of the subject; in still other embodiments, an algorithm may be configured to track the movement and position of the subject's legs to detect proper positioning. Leg position and the angle at which the legs are positioned may be tracked in real-time using, for example, automated segmentation algorithms. When leg position or movement is detected and the movement is determined to be undesirable or not conducive to accurate detection of pulmonary function of the subject, the algorithm may be configured to alarm and/or warn the subject and/or a clinician.

In other cases, the method may comprise coaching related to measuring important, yet subtle, features regarding the subject and the subject's lung function. For example, in some embodiments, the method may further comprise measuring facial, mouth, and lip movement to detect any abnormalities in such movement. In other embodiments, the method may further comprise analyzing the subject's throat to measure levels of relaxation and tightening that are relevant to patient lung function. In still other embodiments, the method may further comprise measuring features and movement of the subject's face, shoulders, chest, to assess whether the subject is applying any accessory muscle usage, which, in some cases, may complicate accurate assessment of the subject's pulmonary function.

In still other cases, the method may comprise coaching in connection with certain quality checks on obtaining the subject's pulmonary function. For example, in some embodiments, the method may further comprise coaching the subject related to conducting quality checks based on lung function tests and/or parameters promulgated by the American Thoracic Society. Such quality checks may entail coaching the subject to inhale for six seconds and obtaining pulmonary function results in connection therewith. In other embodiments, the method may further comprise coaching the subject related to a predefined number (such as two or more or three or more or four or more or five or more) ATS quality efforts, where quality in this context may be defined by ATS standards or other ATS promulgated guidelines or convention.

In still other cases, the method may comprise coaching in connection with a visual display and user interface, such as a visual display used to present results of the subject's pulmonary function testing. In some embodiments, the visual display and user interface comprises a user friendly, visual graph for presentation to both the subject and a technician or a clinician. In other embodiments, the visual display and user interface may be configured to replay chest or body motion, such as, for example, any of the movements of the body not associated with breathing discussed above. In still other embodiments, the visual display and user interface may be configured to display an easily understandable explanation of any errors or areas of improvement relevant to the subject in connection with performing breathing maneuvers and assessing lung function in general.

### Computer Implemented Embodiments

The various method and algorithm steps described in connection with the embodiments disclosed herein can be implemented as electronic hardware, computer software, or combinations of both. To clearly illustrate this interchangeability of hardware and software, various illustrative steps have been described above generally in terms of their functionality. Whether such functionality is implemented as hardware or software depends upon the particular application and design constraints imposed on the overall system applying a method according to the present disclosure. The described functionality can be implemented in varying ways for each particular application, but such implementation decisions should not be interpreted as causing a departure from the scope of the disclosure.

The various illustrative steps, components, and computing systems (such as devices, databases, interfaces, and engines) described in connection with the embodiments disclosed herein can be implemented or performed by a machine, such as a general purpose processor, a graphics processor unit, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor can be a microprocessor, but in the alternative, the processor can be a controller, microcontroller, or state machine, combinations of the same, or the like. A processor can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor can also include primarily analog components. A computing environment can include any type of computer system, including, but not limited to, a computer system based on a microprocessor, a graphics processor unit, a mainframe computer, a digital signal processor, a portable computing device, a personal organizer, a device controller, and a computational engine within an appliance, to name a few.

The steps of a method, process, or algorithm described in connection with the embodiments disclosed herein can be embodied directly in hardware, in a software module executed by a processor, or in a combination of the two. A software module, engine, and associated databases can reside in memory resources such as in RAM memory, FRAM memory, flash memory, ROM memory, EPROM memory, EEPROM memory, registers, hard disk, a removable disk, a CD-ROM, or any other form of non-transitory computer-readable storage medium, media, or physical computer storage known in the art. An exemplary storage medium can be coupled to the processor such that the processor can read information from, and write information to, the storage medium. In the alternative, the storage medium can be integral to the processor. The processor and the storage medium can reside in an ASIC. The ASIC can reside in a user terminal. In the alternative, the processor and the storage medium can reside as discrete components in a user terminal.

As described in detail above, embodiments of the present invention relate to a computer-implemented method for pulmonary function testing, the method comprising, under the control of one or more processing devices: identifying, based on a reference image received from a depth-sensing camera, reference locations of certain features of interest on a subject; determining a chest region of interest comprising a chest area of the subject, based on the location of the features of interest; receiving a plurality of images of the subject from the depth-sensing camera while the subject performs specified breathing maneuvers; generating a three-dimensional representation of the chest region of interest based on the plurality of images of the subject; computing changes in the volume of the chest region of interest based on the three-dimensional representation of the chest; plotting the changes in volume of the chest region of interest on a graph, wherein the graph comprises volume of the chest region of interest over time and wherein certain chest movements are labeled on the graph; filtering the data on the graph using one or more specified filters; generate a flow volume curve based at least in part on rescaling the filtered graph and computing a gradient of the rescaled graph; computing lung function parameters based at least in part on the flow volume curve; and determining potential clinical interpretations of the pulmonary function of the subject based at least in part on the computed lung function parameters.

In some embodiments, the reference image comprises a combination of a plurality of images from one or more depth-sensing cameras. In other embodiments, the certain features of interest on the subject comprise one or more of head or right shoulder or left shoulder or right elbow or left elbow or upper waist or lower waist or right leg or left leg of the subject. In still other embodiments, identifying reference locations of certain features of interest on the subject comprises applying machine learning-driven skeleton tracking. In still other embodiments, determining a chest region of interest is based at least in part on the reference locations of shoulders and lower waist of the subject. In other cases, locations are identified based on pixel coordinates of one or more images received from the depth-sensing camera.

In embodiments, specified breathing maneuvers comprise one or more of normal breathing, a high exertion inhale or a high exertion exhale or an inhale for a specified period of time or an exhale for a specified period of time.

In some cases, embodiments of methods according to the present disclosure further comprise calculating a change in location of one of the certain features of interest in the plurality of images by comparing the location of the one of the certain features of interest in the plurality of images against the reference location of the one of the certain features of interest; determining the change in location of the one of the certain features of interest is based on a movement by the subject other than breathing; and storing information characterizing the change in location with a time stamp. In such cases, determining the change in location of the one of the certain features of interest may be based on a movement by the subject other than breathing comprises determining that a characteristic of a movement exceeds a specified threshold. In such cases, the specified threshold may be a linear distance or an angle. In some cases, the one of the certain features of interest is head of the subject and the movement by the subject other than breathing is neck movement; or the one of the certain features of interest is right shoulder or left shoulder of the subject and the movement by the subject other than breathing is shrugging or rocking; or the one of the certain features of interest is right shoulder joint or left shoulder joint of the subject and the movement by the subject other than breathing is a side-to-side movement; or the one of the certain features of interest is right knee or left knee or right ankle or left ankle of the subject and the movement by the subject other than breathing is a bad leg position.

In embodiments of methods according to the present disclosure, computing changes in the volume of the chest region of interest based on the three-dimensional representation of the chest comprises: determining right and left shoulder locations and right and left waist locations of the subject based on the plurality of images of the subject; assigning the boundaries of the chest region of interest to be the width from the right to left shoulder of the subject and a specified percentage of the height between the shoulder and the waist of the subject; generating a three-dimensional triangulated mesh representation of the chest region of interest; computing a volume of the chest region of interest by summing a volume of each of a plurality of three-dimensional pixels that comprise the three-dimensional triangulated mesh representation of the chest region of interest. In such cases, generating a three-dimensional triangulated mesh representation of the chest region of interest comprises applying a Delaunay algorithm.

Some embodiments may further comprise assessing the quality of effort of the subject performing the specified breathing maneuvers by identifying one or more of: sub-maximal inhalation and exhalation; or hesitation before initial blasting; or coughing; or air leaking; or extra breaths; or accessory muscle usage. In such cases, embodiments may further comprise generating a lung function graph that reflects the assessment of the quality of effort of the subject performing the specified breathing maneuvers.

In some cases, embodiments further comprise training a model to predict lung function parameters from the plurality of images of the subject from the depth-sensing camera while the subject performs specified breathing maneuvers as well as certain clinical information about the subject. In such embodiments training the model may comprise: obtaining spirometer data paired with images from the depth-sensing camera for a plurality of subjects; dividing the paired data into a first group of data for training the model and a second group of data for testing the model; receiving certain clinical information about the subjects; using the first group of data and the certain clinical information to train the model to predict lung function parameters; and validating the performance of the trained model using the second group of data and the certain clinical information. In such cases, the certain clinical information may comprise one or more of body mass index, height, chest circumference or medical history. In some embodiments, the model is a deep learning model. In such cases, the deep learning model comprises a convolutional neural network-based architecture. In other cases, the model is a machine learning model. In such cases, the machine learning model may comprise a multi-linear regression model.

In embodiments, filtering the data on the graph using one or more specified filters comprises: estimating a trend line by computing a least-squares regression line based on the graph; and subtracting the estimated trend line from the graph.

In other embodiments, filtering the data on the graph using one or more specified filters comprises applying Savgol filtering to the graph. In such cases, Savgol filtering may comprise using a least-square to fit a polynomial to the graph while maintaining the shape of the data.

In some embodiments, the subject methods further comprise identifying sections of interest of the filtered graph. In such cases, the sections of interest of the filtered graph comprise periods of one or more of tidal breathing or inhalation or exhalation.

In embodiments, rescaling the filtered graph comprises changing the scale of the graph from units based on depth-sensor camera to lung volume units. In other embodiments, the lung function parameters comprise one or more of forced exhalatory volume (FEV), or forced exhalatory volume in one second (FEV1), or forced exhalatory volume in six seconds (FEV6), or forced vital capacity (FVC). In still other embodiments, subject methods may further comprise displaying the flow volume curve and associated lung function parameters.

### Systems for Pulmonary Function Testing

As summarized above, aspects of the present disclosure include systems for pulmonary function testing. Systems according to certain embodiments comprise a first depth-sensing camera configured to generate depth-sensing images of a subject; and a processor comprising memory operably coupled to the processor, wherein the memory comprises instructions stored thereon, which, when executed by the processor, cause the processor to execute steps corresponding to the subject methods described herein; and an operable connection between the depth-sensing camera and the processor.

In some embodiments of systems according to the present disclosure the system comprises: a first depth-sensing camera configured to generate depth-sensing images of a subject; and a processor comprising memory operably coupled to the processor, wherein the memory comprises instructions stored thereon, which, when executed by the processor, cause the processor to: identify, based on a reference image received from the depth-sensing camera, reference locations of certain features of interest on the subject; determine a chest region of interest comprising a chest area of the subject, based on the location of the features of interest; receive a plurality of images of the subject from the depth-sensing camera while the subject performs specified breathing maneuvers; generate a three-dimensional representation of the chest region of interest based on the plurality of images of the subject; compute changes in the volume of the chest region of interest based on the three-dimensional representation of the chest; plot the changes in volume of the chest region of interest on a graph, wherein the graph comprises volume of the chest region of interest over time and wherein certain chest movements are labeled on the graph; filter the data on the graph using one or more specified filters; generate a flow volume curve based at least in part on rescaling the filtered graph and computing a gradient of the rescaled graph; compute lung function parameters based at least in part on the flow volume curve; and determine potential clinical interpretations of the pulmonary function of the subject based at least in part on the computed lung function parameters; and an operable connection between the depth-sensing camera and the processor.

In other embodiments of systems according to the present disclosure, the system may further comprise a second depth-sensing camera configured to generate depth-sensing images of the subject. Such second depth-sensing camera may be positioned in a different location, such as at a different distance from the subject, or a different angle of orientation towards the subject, than the first camera. That is, the first and second cameras may be utilized to obtain to different and distinct views of the subject by orienting each to obtain a different field of view.

Any convenient depth sensing camera may be used in embodiments of the subject systems. For example, any off the shelf, commercially available depth sensing camera, or equivalents thereto, such as those discussed above.

In instances, the processor and/or memory may be operably connected to the first depth sensing camera and in some cases, the second depth sensing camera. Such operable connection may take any convenient form such that images generated by either depth sensing camera may be obtained by the processor by any convenient input technique, such as via a wired or wireless network connection, shared memory, a bus or similar communication protocol with a source of cytometric data, such as an ethernet connection or a Universal Serial Bus (USB) connection, portable memory devices or the like.

In embodiments, the memory comprises further instructions stored thereon, which, when executed by the processor, cause the processor to cause volume curves or lung function curves or analyses or interpretations of the same to be displayed on a display device. Any convenient display device, such as a liquid crystal display (LCD), light-emitting diode (LED) display, plasma (PDP) display, quantum dot (QLED) display or cathode ray tube display device. The processor and/or memory may be operably connected to the display device, for example, via a wired, such as a Universal Serial Bus (USB) connection, or wireless connection, such as a Bluetooth connection.

### Utility

The subject methods and systems find use in a variety of applications where it is desirable to measure and assess pulmonary lung function. In some embodiments, the methods and systems described herein find use in clinical settings such as any clinical setting where traditional spirometer-based pulmonary function testing may be applied. In other embodiments, the methods and systems described herein find use in remote medicine settings, where pulmonary function testing may be newly enabled by application of the present methods and systems, such as in telemedicine contexts. In addition, the subject methods and systems find use in improving the effectiveness and accuracy of measuring a subject's pulmonary function. In some cases, the subject methods and systems find use in improving user friendliness of a system for determining pulmonary function by including additional interactive and intuitive functionality. In some cases, the subject methods and systems find use in improving diagnosis of a subject's pulmonary condition.

The following is offered by way of illustration and not by way of limitation.

### Experimental

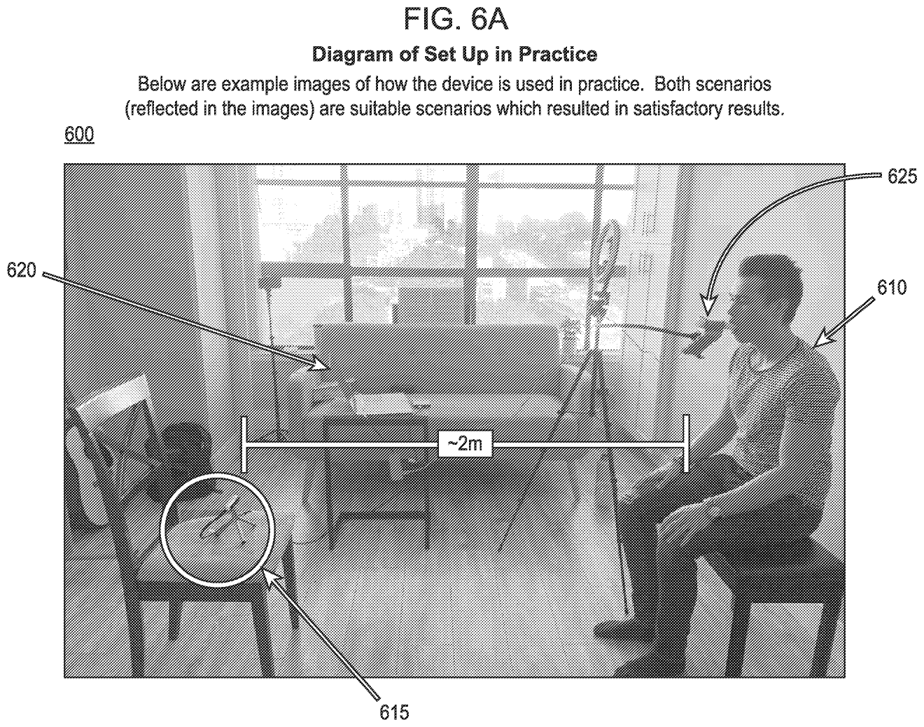

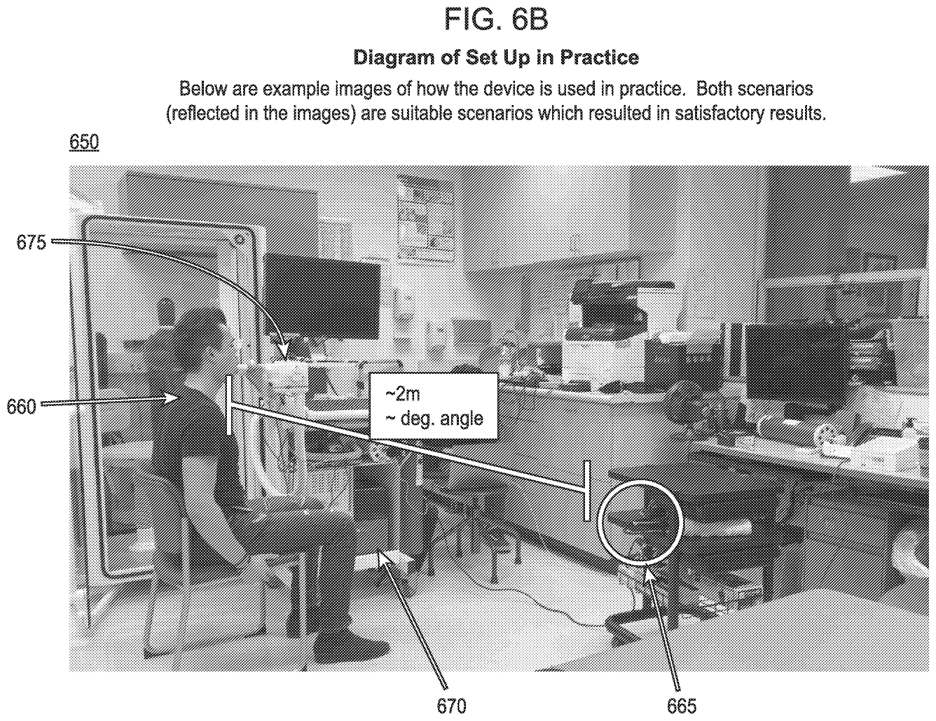

FIG. 6A depicts an example of a subject 610 utilizing a system 600 according to the present disclosure to assess the subject's 610 pulmonary lung function. FIG. 6A shows the system 600 in use in a home environment to demonstrate the applicability of the system in connection with, for example, telemedicine applications. FIG. 6A shows a subject 610 facing a depth sensing camera 615 such that the depth sensing camera takes depth-based, three-dimensional images of subject 610 as the subject performs breathing maneuvers. Depth sensing camera is positioned approximately two meters from subject 610 and approximately “head on” to the subject, such that subject's 610 shoulders, waist and chest region are clearly in the field of view of camera 615. Processor and memory 620 with instructions thereon are configured to apply an embodiment of the subject methods. Subject 610 is seen engaging with spirometer 625. Spirometer 625 need not comprise an aspect of embodiments of systems according to the present disclosure but may be used to validate results of pulmonary function assessment obtained by the subject methods and systems.

FIG. 6B depicts an example of a subject 660 utilizing a system 650 according to the present disclosure to assess the subject's 660 pulmonary lung function. FIG. 6B shows the system 650 in use in a clinical environment to demonstrate the applicability of the system in connection with traditional medical clinic applications. FIG. 6B shows a subject 650 facing a depth sensing camera 665 such that the depth sensing camera takes depth-based, three-dimensional images of subject 650 as the subject performs breathing maneuvers. Depth sensing camera is positioned approximately two meters from subject 650 at approximately a twenty degree angle from the subject's 650 forward facing direction. Positioning camera 665 at a twenty degree angle enables camera 665 to obtain a view of subject's 610 shoulders, waist and chest region while also providing some information about the subject's 665 side and back features. Processor and memory 670 with instructions thereon are configured to apply an embodiment of the subject methods. Subject 660 is seen engaging with spirometer 675. Spirometer 675 need not comprise an aspect of embodiments of systems according to the present disclosure but may be used to validate results of pulmonary function assessment obtained by the subject methods and systems.

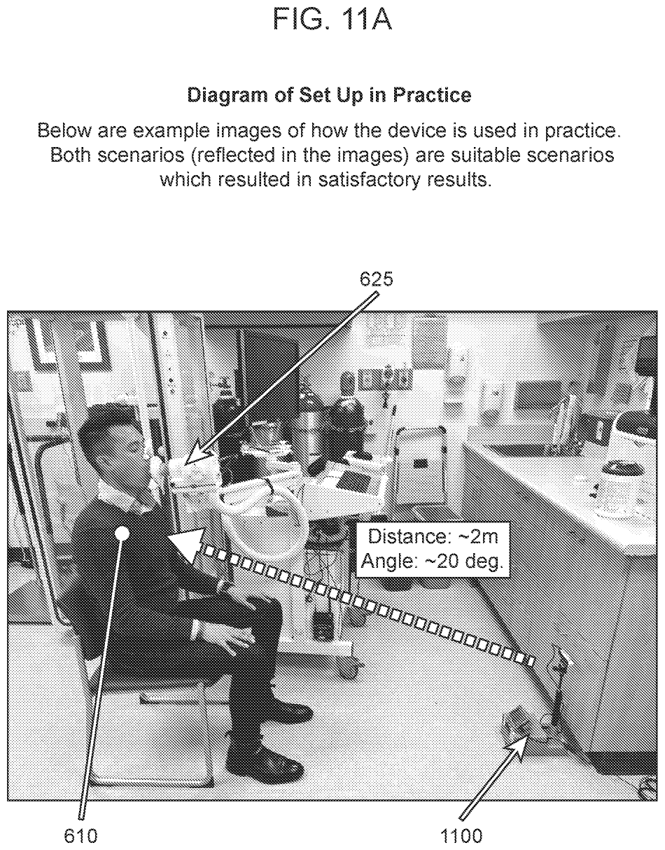

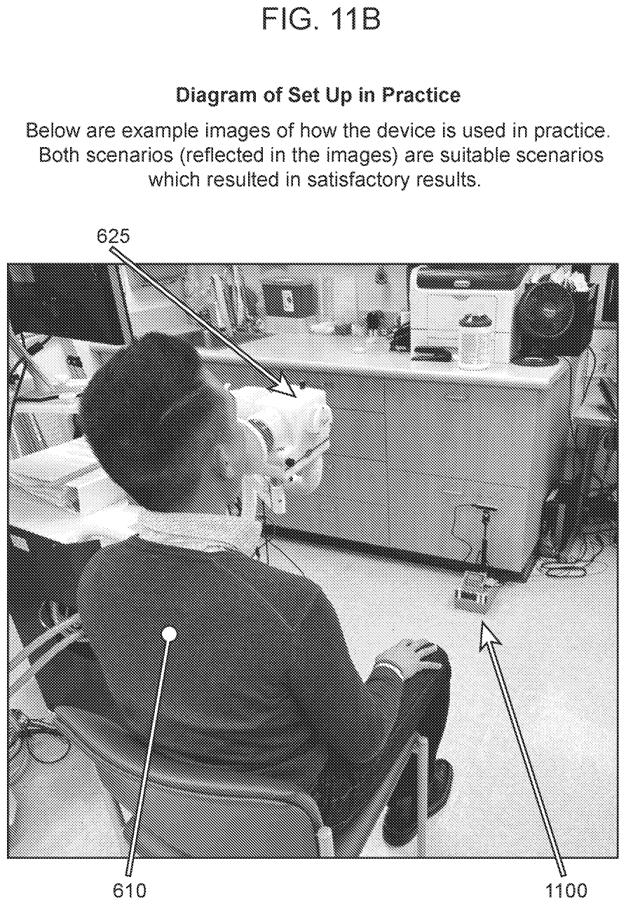

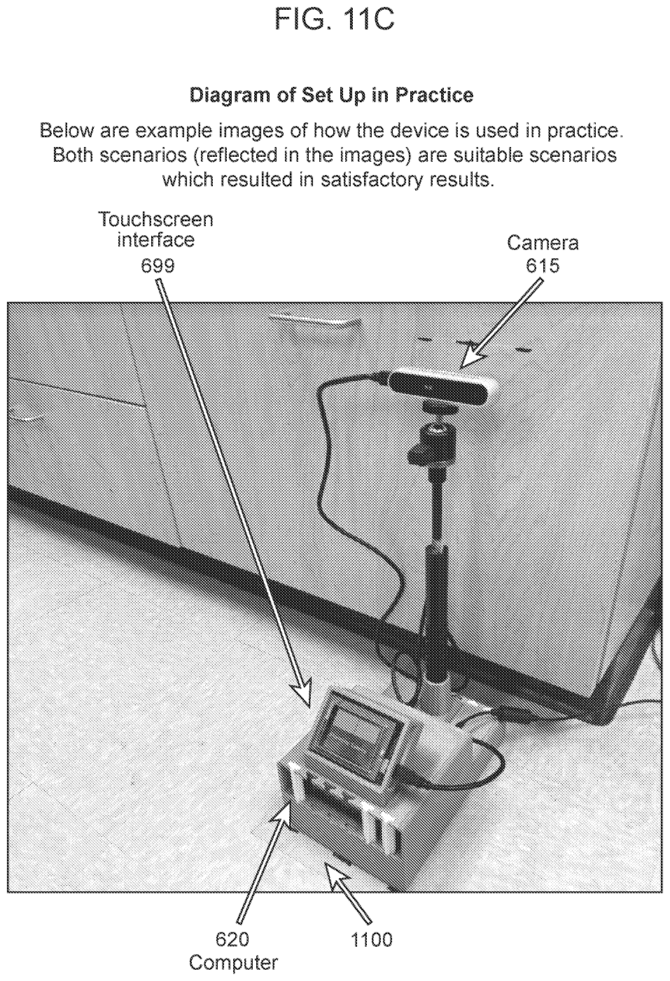

FIGS. 11A, 11B and 11C depict an example of a subject utilizing a system according to the present disclosure to assess the subject's pulmonary lung function where the system is a variation of that depicted in FIGS. 6A and 6B. Identical labels and aspects of the experimental set-up seen in FIGS. 6A and 6B are not separately described in connection with FIGS. 11A, 11B and 11C. FIG. 11A depicts an example of a subject 610 utilizing a system 1100 that is an exemplary embodiment of a system according to the present disclosure to assess the subject's 610 pulmonary lung function.

FIG. 11A shows a subject 610 facing exemplary system 1100 that includes depth sensing camera such that the depth sensing camera takes depth-based, three-dimensional images of subject 610 as the subject performs breathing maneuvers. Depth sensing camera of exemplary system 1100 is positioned approximately two meters from subject 610 and approximately “head on” to the subject, such that subject's 610 shoulders, waist and chest region are clearly in the field of view of depth sensing camera of exemplary system 1100. Depth sensing camera of exemplary system 1100 is positioned at an angle of approximately 20 degrees below subject 610. Subject 610 is seen engaging with spirometer 625. Spirometer 625 need not comprise an aspect of embodiments of systems according to the present disclosure but may be used to validate results of pulmonary function assessment obtained by the subject methods and systems.

FIG. 11B shows another view of subject 610 facing exemplary system 1100 that includes depth sensing camera such that the depth sensing camera takes depth-based, three-dimensional images of subject 610 as the subject performs breathing maneuvers. FIG. 11B depicts a view of subject 610 and exemplary system 1100 from behind subject's head.

FIG. 11C shows a close up view of exemplary system 1100 that includes depth sensing camera 615, computer 620 as well as touchscreen interface to computer 620. Exemplary system 1100 is configured such that camera 615 is mounted on a mast above computer 620 and touchscreen 699 so that system 1100 can be positioned on the floor in front of subject 610 and the height, orientation and angle of camera 615 is configurable.

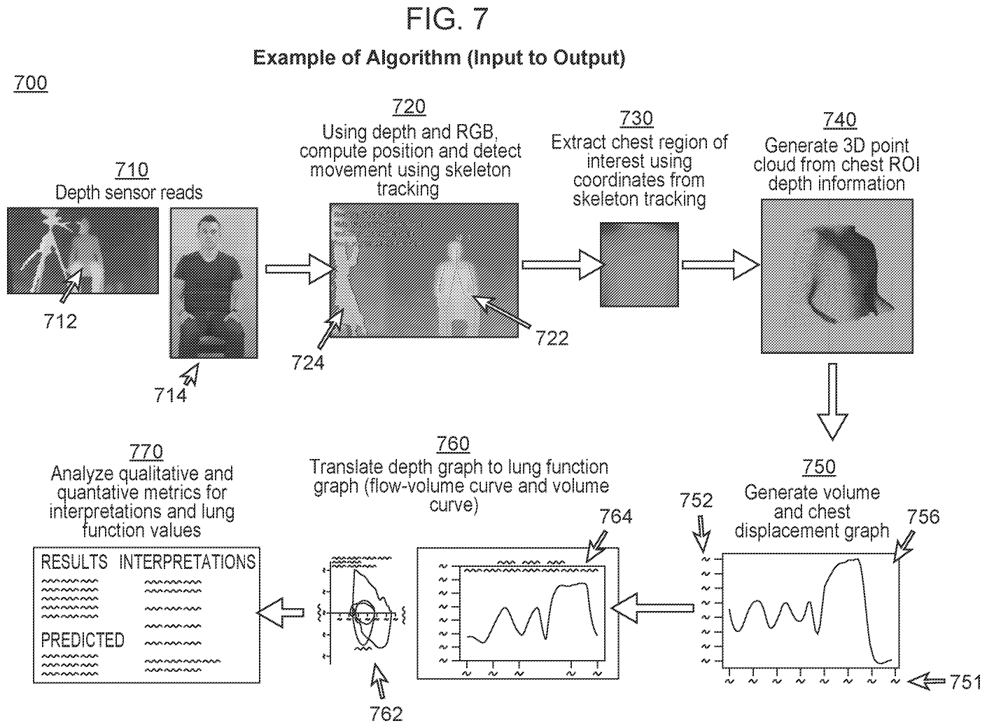

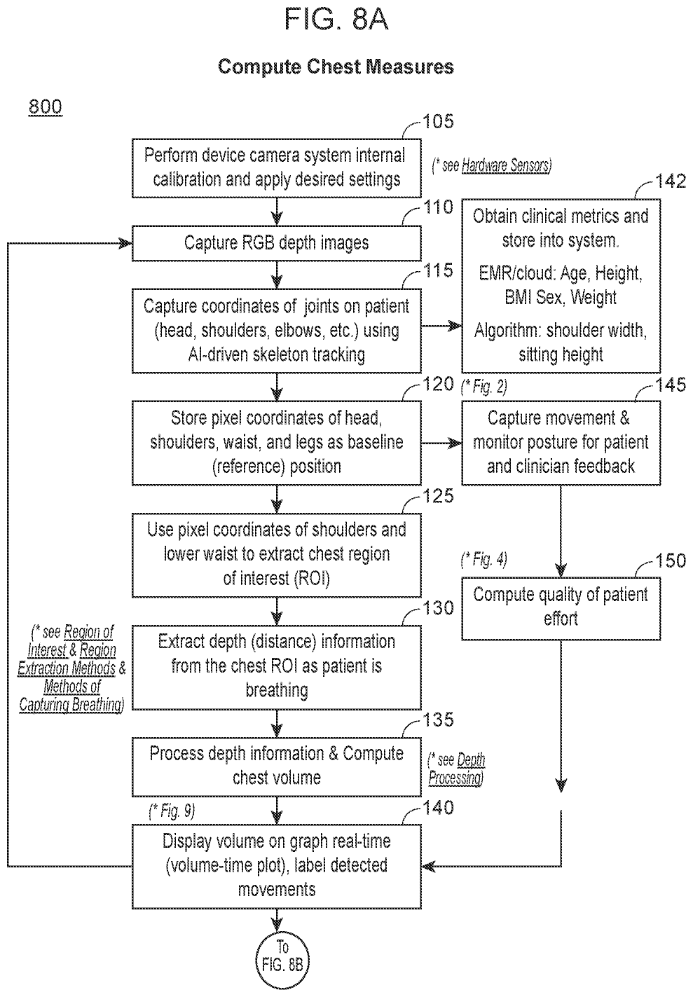

FIG. 7 depicts an example application of an embodiment of a subject method 700 for pulmonary function testing of a subject according to the present disclosure.

At step 710, a depth sensing camera and an RGB (red green blue) camera capture a depth image 712 and an RGB image 714 of subject. The depth image 712 shows aspects of subject that are further away from the camera in a lighter shade than those aspects of the subject that are closer to the camera. A chest region of interest 716 of subject is detected and highlighted with a dotted line in RGB image 714.

At step 720, key points, such as a chest region of interest 716 are identified and located using pixel coordinates in the depth sensing image. A skeleton model 722 is superimposed onto the depth image. Movement of the subject is detected and information about the subject's movements 724 is displayed on the screen for review by the subject or a clinician.

At step 730, a chest region of interest is extracted from the depth image based on the coordinates of the chest region of interest obtained at step 720.

At step 740, a three dimensional point cloud representation of the chest region of interest is generated from the chest region of interest depth information obtained at step 720 and step 730.

At step 750, the three dimensional point cloud representation of the chest region of interest is used to calculate chest volume information—or chest displacement—over time. The calculated chest volumes 752 plotted against time 754 as the subject breathes or performs breathing maneuvers. The results are summarized in graph 756 of chest volume over time.

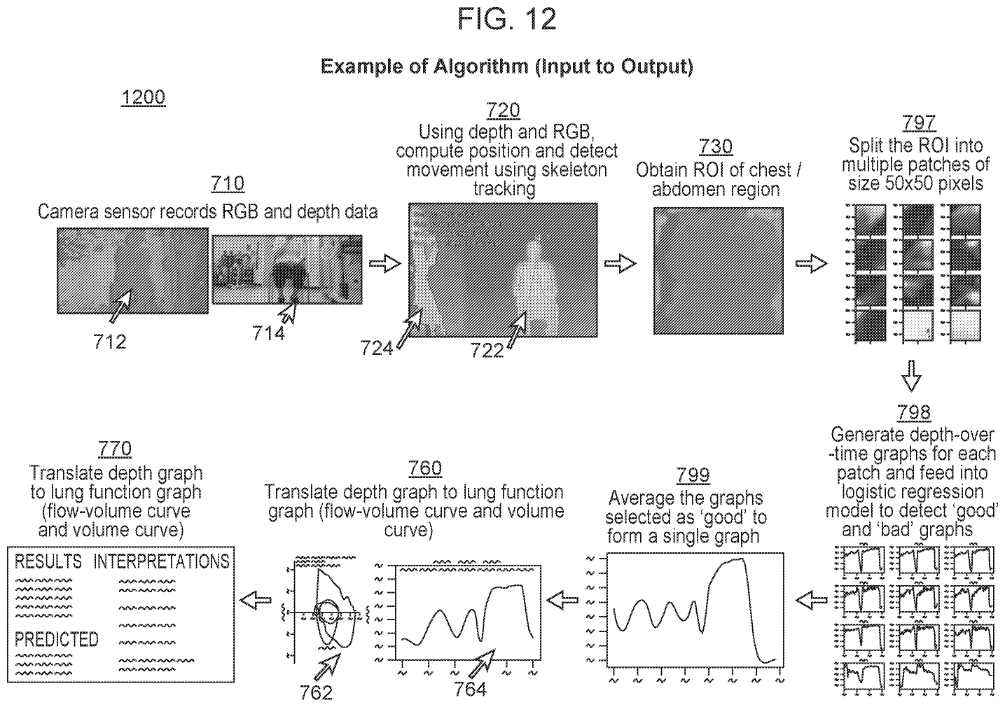

In other embodiments, at step 730, the process proceeds to step 797 as depicted in alternative embodiment of algorithm 1200 according to the present invention in FIG. 12.

At step 797, the image obtained of the region of interest (ROI) is split into multiple patches, in this case, multiple patches of size 50×50 pixels each. In alternative embodiment 1200, algorithm 1200 proceeds to step 798.

At step 798, depth-over-time graphs for each patch are generated and fed into logistic regression model to detect ‘good’ and ‘bad’ graphs. In alternative embodiment 1200, algorithm 1200 proceeds to step 799.

At step 799, the graphs selected as ‘good’ in step 798 are averaged together to form a single graph seen in step 799. In alternative embodiment 1200, algorithm 1200 proceeds to step 760.

At step 760, the graph of chest displacement over time is translated into lung function graphs. In particular, at step 760, the graph is translated into a flow volume curve 762 (i.e., a plot of flow or rate of change of the volume of air displaced by the subject versus the volume of air taken into the subject's lungs) and a volume curve 764, showing lung volume over time.

At step 770, quantitative and qualitative metrics resulting from analysis of the lung function curves generated at step 760 are displayed on a user interface for consumption by the subject or a clinician.

FIG. 12 depicts an example application of an embodiment of a subject method 1200 for pulmonary function testing of a subject according to the present disclosure and is a variation of that shown in FIG. 7. Identical labels and aspects of the experimental set-up seen in FIG. 7 are not separately described in connection with FIG. 12. As described above, FIG. 12 includes steps 797, 798 and 799, in each case, as described above, which in some cases, in exemplary embodiment of algorithm 1200, follow block 730.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112(f) or 35 U.S.C. § 112(6) is not invoked.

What is claimed is:

1. A computer-implemented method for pulmonary function testing, the method comprising, under the control of one or more processing devices:
identifying, based on a reference image received from a depth-sensing camera, reference locations of certain features of interest on a subject;
determining a chest region of interest comprising a chest area of the subject, based on the location of the features of interest;
receiving a plurality of images of the subject from the depth-sensing camera while the subject performs specified breathing maneuvers;
generating a three-dimensional representation of the chest region of interest based on the plurality of images of the subject;
computing changes in the volume of the chest region of interest based on the three-dimensional representation of the chest;
plotting the changes in volume of the chest region of interest on a graph, wherein the graph comprises volume of the chest region of interest over time and wherein certain chest movements are labeled on the graph;
filtering the data on the graph using one or more specified filters; generate a flow volume curve based at least in part on rescaling the filtered graph and computing a gradient of the rescaled graph;
computing lung function parameters based at least in part on the flow volume curve; and
determining potential clinical interpretations of the pulmonary function of the subject based at least in part on the computed lung function parameters.

2. The computer-implemented method of claim 1, wherein the reference image comprises a combination of a plurality of images from one or more depth-sensing cameras.

3. The computer-implemented method of any of the previous claims, wherein the certain features of interest on the subject comprise one or more of head or right shoulder or left shoulder or right elbow or left elbow or upper waist or lower waist or right leg or left leg of the subject.

4. The computer-implemented method of any of the previous claims, wherein identifying reference locations of certain features of interest on the subject comprises applying machine learning-driven skeleton tracking.

5. The computer-implemented method of any of the previous claims, wherein determining a chest region of interest is based at least in part on the reference locations of shoulders and lower waist of the subject.

6. The computer-implemented method of any of the previous claims, wherein locations are identified based on pixel coordinates of one or more images received from the depth-sensing camera.

7. The computer-implemented method of any of the previous claims, wherein specified breathing maneuvers comprise one or more of normal breathing, a high exertion inhale or a high exertion exhale or an inhale for a specified period of time or an exhale for a specified period of time.

8. The computer-implemented method of any of the previous claims, further comprising:
calculating a change in location of one of the certain features of interest in the plurality of images by comparing the location of the one of the certain features of interest in the plurality of images against the reference location of the one of the certain features of interest;
determining the change in location of the one of the certain features of interest is based on a movement by the subject other than breathing; and
storing information characterizing the change in location with a time stamp.

9. The computer-implemented method of any of claim 8, wherein determining the change in location of the one of the certain features of interest is based on a movement by the subject other than breathing comprises determining that a characteristic of a movement exceeds a specified threshold.

10. The computer-implemented method of any of claim 9, wherein the specified threshold is a linear distance or an angle.

11. The computer-implemented method of any of claims 8-10, wherein
the one of the certain features of interest is head of the subject and the movement by the subject other than breathing is neck movement; or
the one of the certain features of interest is right shoulder or left shoulder of the subject and the movement by the subject other than breathing is shrugging or rocking; or
the one of the certain features of interest is right shoulder joint or left shoulder joint of the subject and the movement by the subject other than breathing is a side-to-side movement; or
the one of the certain features of interest is right knee or left knee or right ankle or left ankle of the subject and the movement by the subject other than breathing is a bad leg position.

12. The computer-implemented method of any of the previous claims, wherein computing changes in the volume of the chest region of interest based on the three-dimensional representation of the chest comprises:
determining right and left shoulder locations and right and left waist locations of the subject based on the plurality of images of the subject;
assigning the boundaries of the chest region of interest to be the width from the right to left shoulder of the subject and a specified percentage of the height between the shoulder and the waist of the subject;
generating a three-dimensional triangulated mesh representation of the chest region of interest;
computing a volume of the chest region of interest by summing a volume of each of a plurality of three-dimensional pixels that comprise the three-dimensional triangulated mesh representation of the chest region of interest.

13. The computer-implemented method of claim 12, wherein generating a three-dimensional triangulated mesh representation of the chest region of interest comprises applying a Delaunay algorithm.

14. The computer-implemented method of any of the previous claims, further comprising assessing the quality of effort of the subject performing the specified breathing maneuvers by identifying one or more of:
sub-maximal inhalation and exhalation; or
hesitation before initial blasting; or
coughing; or
air leaking; or
extra breaths; or
accessory muscle usage.

15. The computer-implemented method of claim 14, further comprising generating a lung function graph that reflects the assessment of the quality of effort of the subject performing the specified breathing maneuvers.

16. The computer-implemented method of any of the previous claims, further comprising training a model to predict lung function parameters from the plurality of images of the subject from the depth-sensing camera while the subject performs specified breathing maneuvers as well as certain clinical information about the subject.

17. The computer-implemented method of claim 16 wherein training the model comprises:
obtaining spirometer data paired with images from the depth-sensing camera for a plurality of subjects;
dividing the paired data into a first group of data for training the model and a second group of data for testing the model;
receiving certain clinical information about the subjects;
using the first group of data and the certain clinical information to train the model to predict lung function parameters; and
validating the performance of the trained model using the second group of data and the certain clinical information.

18. The computer-implemented method of claim 17 wherein the certain clinical information comprises one or more of body mass index, height, chest circumference or medical history.

19. The computer-implemented method of any of claims 17-18 wherein the model is a deep learning model.

20. The computer-implemented method of claim 19 wherein the deep learning model comprises a convolutional neural network-based architecture.

21. The computer-implemented method of any of claims 17-18 wherein the model is a machine learning model.

22. The computer-implemented method of claim 21 wherein the machine learning model comprises a multi-linear regression model.

23. The computer-implemented method of any of the previous claims wherein filtering the data on the graph using one or more specified filters comprises:
estimating a trend line by computing a least-squares regression line based on the graph; and
subtracting the estimated trend line from the graph.

24. The computer-implemented method of any of the previous claims wherein filtering the data on the graph using one or more specified filters comprises applying Savgol filtering to the graph.

25. The computer-implemented method of claim 24 wherein Savgol filtering comprises using a least-square to fit a polynomial to the graph while maintaining the shape of the data.

26. The computer-implemented method of any of the previous claims further comprising identifying sections of interest of the filtered graph.

27. The computer-implemented method of claim 26 wherein the sections of interest of the filtered graph comprise periods of one or more of tidal breathing or inhalation or exhalation.

28. The computer-implemented method of any of the previous claims wherein rescaling the filtered graph comprises changing the scale of the graph from units based on depth-sensor camera to lung volume units.

29. The computer-implemented method of any of the previous claims wherein the lung function parameters comprise one or more of forced exhalatory volume (FEV), or forced exhalatory volume in one second (FEV1), or forced exhalatory volume in six seconds (FEV6), or forced vital capacity (FVC).

30. The computer-implemented method of any of the previous claims further comprising displaying the flow volume curve and associated lung function parameters.

31. A system for pulmonary function testing, the system comprising:
a first depth-sensing camera configured to generate depth-sensing images of a subject; and
a processor comprising memory operably coupled to the processor, wherein the memory comprises instructions stored thereon, which, when executed by the processor, cause the processor to:
identify, based on a reference image received from the depth-sensing camera, reference locations of certain features of interest on the subject;
determine a chest region of interest comprising a chest area of the subject, based on the location of the features of interest;
receive a plurality of images of the subject from the depth-sensing camera while the subject performs specified breathing maneuvers;
generate a three-dimensional representation of the chest region of interest based on the plurality of images of the subject;
compute changes in the volume of the chest region of interest based on the three-dimensional representation of the chest;
plot the changes in volume of the chest region of interest on a graph, wherein the graph comprises volume of the chest region of interest over time and wherein certain chest movements are labeled on the graph;
filter the data on the graph using one or more specified filters; generate a flow volume curve based at least in part on rescaling the filtered graph and computing a gradient of the rescaled graph;
compute lung function parameters based at least in part on the flow volume curve; and
determine potential clinical interpretations of the pulmonary function of the subject based at least in part on the computed lung function parameters; and
an operable connection between the depth-sensing camera and the processor.

32. The system for pulmonary function testing according to claim 31, further comprising:
a second depth-sensing camera configured to generate depth-sensing images of the subject.
